(19) United States
(12) Patent Application Publication
(10) Pub. No.: US 2022/0175859 A1
(43) Pub. Date: Jun. 9, 2022

(54) Tetraselmis Extract

(71) Applicant: SYMRISE AG, Holzminden (DE)

(72) Inventors: Martina Herrmann, Hameln (DE); Sandra Gaebler, Hoexter (DE); Dominik Stuhlmann, Holzminden (DE); Ann-Christin Weseloh, Polle (DE); Imke Meyer, Bodenwerder (DE)

(21) Appl. No.: 16/976,023

(22) PCT Filed: Feb. 27, 2019
(86) PCT No.: PCT/EP2019/054914

§ 371 Date: Aug. 26, 2020

(30) Foreign Application Priority Data
Feb. 28, 2018 (EP) PCT/EP2018/054985

(51) Int. Cl. A61K 36/05; A61K 31/455; A61K 8/9722; A61K 45/06; A61Q 19/00; A61Q 5/00

(52) U.S. Cl. CPC A61K 36/05; A61K 31/455; A61K 8/9722; A61Q 17/04; A61Q 19/00; A61Q 5/00; A61K 2236/333; A61K 45/06

(57) ABSTRACT

The present invention relates to a novel extract and a method for obtaining such an effective Tetraselmis extract which was found to have advantages in dermatological treatments and can be employed as a topical cosmetic.

The present invention relates to a novel Tetraselmis extract and a method for obtaining this Tetraselmis extract which was found to have advantages in dermatological treatments and can be employed as a topical cosmetic.

Document EP 2 193 785 A2 is concerned with the extraction of Tetraselmis suecica, but the process is conducted at low temperatures. These extracts have different compositions, in particular with regard to the amino acid and sugar components. Surprisingly it was found that an extract with this composition has an impact on the skin treatment properties of topical applications employing the Tetraselmis suecica extract.

Sebaceous glands (SGs) are skin appendages found everywhere on the body's skin except the palms of the hands and the soles and dorsum of the feet. A sebaceous gland consists of one or more lobules within the same gland. SGs secret a natural oil, called sebum, which participates with the sweat to compose the hydrolipidic film that covers the skin. Human sebum is a complex mixture of approx. 40-60% triglycerides, diglycerides and free fatty acids, 25-30% wax esters, 12-15% squalene, 3-6% cholesterol esters, and 1.5-2.5% cholesterol.

In its role as a barrier to environmental stress, including environmental toxic agents and UV light, the skin is supported by the SGs and sebum has important functions for healthy status and appearance of the skin. It plays a role in barrier protection and maintenance, especially regulation of transepidermal water loss, protection of skin and hair against friction, maintenance of the skin biofilm and delivery of antioxidants (squalene, coenzyme Q10, and vitamin E) to the skin surface. Furthermore, it is involved in epidermal development, body odor, and generation of pheromones. Sebum is directly involved in hormonal signaling, epidermal differentiation, and protection from ultraviolet (UV) radiation. It also modulates composition and proliferation of the natural micro-flora of the skin.

There are two types of SGs, those connected to hair follicles, in pilosebaceous units, and those that exist independently (not associated with hair follicles). When they are associated to the hair follicles, one or more glands may surround each hair follicle, and the glands themselves are surrounded by arrector pili muscles. SGs are particularly abundant on the face, the scalp and in the midline of the back. They can number up to 400-900 glands/cm² on the face. They are also found in hairless areas (glabrous skin) of the eyelids (called meibomian glands), ears (ceruminous glands), nose, penis, labia minora, the inner oral mucosa of the cheek (Fordyce's spots), and nipples.

Sebocytes are the major cells within the SGs. Their purpose is the production and secretion of the sebum via the differentiation and disintegration of fully mature cells, a unique process termed holocrine secretion. The sebocytes may be classified into undifferentiated cells arranged in a single layer facing the basal lamina. They bear characteristics of stem cells, since they give rise to a continual flux of proliferating and differentiating cells. Growing towards the center of the gland lobules, the basal cells gradually differentiate into an early differentiated cell type, an advanced differentiated cell type, a fully differentiated cell type and the mature sebocyte. Characteristically the accumulation of lipids in the cytoplasm of the sebocytes increases with advanced differentiation. In fully differentiated and in mature sebocytes, the nuclei become distorted and disintegrated and the cells rupture. The sebum drains into the sebaceous duct and is then released into the hair follicle around the hair shaft. Once secreted, the sebum is colonized by various xenobiotes, whose development is controlled by several defensive mechanisms and by the contact with ambient oxygen. Oxygen and micro-organisms transform “native” sebum, lysis of triglycerides to fatty acids being the most pronounced activity.

Sebocytes possess an enzymatic machinery competent for the synthesis of all the lipid classes present in the sebum. Sebum fatty acids are characterized by a large diversity including linear and branched species with odd or even carbon number, long chain, and unusual unsaturation. Acetate, propionate, isobutyrate, isovalerate, and 2-methyl-butyrate are used to produce the different fatty acids by extension with the addition of two-carbon moieties derived from the malonyl-CoA. Desaturation occurs by the activity of the Δ6-desaturase (fatty acid desaturase 2) and Δ9-desaturase (Stearoyl-CoA desaturase).

Linoleic acid is considered to be directly involved in the sebaceous lipid synthesis and to be incorporated in the epidermal lipids of the infundibulum. Through the activation of β-oxidation, linoleic acid is transformed into two-carbon precursors, which yield acetyl-CoA, the starter of the biosynthetic pathway. The latter leads to squalene and wax esters formation. Since linoleic acid is an essential fatty acid, its plasma levels likely regulate its concentration in the sebocytes. Fatty acids are subsequently used to synthesize triglycerides, cholesterol, and wax esters.

Triglycerides are synthesized from fatty acids and glycerol. Monoacylglycerol acyltransferase (MGAT) enzymes convert monoacylglycerol to diacylglycerol, which is the penultimate step in one pathway for triacylglycerol synthesis. Acyl CoA/diacylglycerol acyltransferases (DGAT) 1 and 2 are the key enzymes that catalyze the final step in the triglyceride synthesis. Wax esters are produced in a two-step process involving a fatty-acyl-CoA reductase and wax synthase enzymes. Saturated fatty acids are preferentially included over its monounsaturated. The enzymes acyl-CoA cholesterol acyltransferase 1 (ACAT 1) is highly expressed in the SG, where it allows for the incorporation of cholesteryl esters into cytoplasmic lipid droplets. Cholesterol and squalene share the initial steps of their biosynthesis. Squalene is the last linear intermediate in cholesterol biosynthesis.

Liver-X receptors (LXRs), which are members of the NHR family play a critical role in cholesterol homeostasis and lipid metabolism. Treatment of SZ95 sebocytes with the LXR ligands enhanced accumulation of lipid droplets in the cells which could be explained through induction of the expression of the LXRα receptor and known LXR targets, such as fatty acid synthase (FASN) and sterol regulatory element-binding protein-1 (SREBP-1).

Peroxisome proliferator-activated receptors (PPAR) are members of the nuclear hormone receptor (NHR) family and act as transcriptional regulators of a variety of genes including those involved in lipid metabolism in skin. Various fatty acids, eicosanoids, and prostanoids (comprising prostaglandins, prostacyclins, and thromboxanes) activate PPARs. PPARs are expressed in human SGs and in human SZ95 sebocytes. PPARγ activates sebocyte development (proliferation) and lipogenesis. PPARγ is involved in oxidative stress mediated prostaglandin E2 production and induces COX-2 expression in human SZ95 sebocytes.

Prostaglandins are lipid mediators synthesized in response to numerous growth factors and environmental stimuli. The production of prostaglandins is dependent on the activity of cyclooxygenase enzymes (COX-1 and COX-2). Sebocytes produce cyclooxygenase 2 (COX-2), also termed prostaglandinsynthase-2 (PGHS-2), in vivo and in vitro. The importance of COX-2 in sebaceous gland development is seen in transgenic mice with targeted overexpression of the inducible COX-2 isoform. These mice develop sebaceous gland hyperplasia, increased sebum production, and greasy hair suggesting an important role for COX-2 and prostaglandins in sebocyte proliferation, and lipid metabolism.

The function of SGs is controlled by various other factors such as e.g. growth factors. Insulin-like growth factor 1 (IGF-1) plays a key role in the induction of lipid synthesis in human sebocytes. IGF-1 increases lipogenesis by inducing SREBP-1 which preferentially regulates genes of fatty acid synthesis.

High rates of sebum production per sebocyte result in low levels of linoleate in the sebaceous esters, subjecting the follicular epithelium to essential fatty acid deficiency and the characteristic hyperkeratosis that results in comedo formation. Suppression of sebum production by drugs elevates sebum linoleate concentration and relieves follicular hyperkeratosis.

Undesirable hyperactivity of sebaceous glands occurs e.g. on the face. Here the overproduction of sebum gives the skin a shiny, greasy and aesthetically undesirable appearance (oily skin) frequently accompanied by large pores. A demographic worldwide study revealed that oily skin is a common concern of 70% of US women and 62% of Japanese women. Excess sebum blocks pores, provides nourishment to bacteria that live upon the skin. It can promote other slight blemishes, such as comedones. In some cases, more serious disorders can occur in the presence of excessive sebum, such as acne. Acne vulgaris is the most common inflammatory skin condition that affects up to 85% of adolescents, and frequently persists into adulthood. The pathogenesis is multifactorial and includes sebaceous gland overactivity; sebum excretion rate correlates with acne severity and predicts acne outcome. Sebaceous glands are relatively anoxic and support the growth of facultative anaerobes such as Propionibacterium acnes which plays an important role in acne and its density increases with increased sebum excretion rate.

Enlarged skin pores refer to conditions that present with visible topographic changes of skin surfaces. Although not a medical concern, enlarged pores are a cosmetic concern for a large number of individuals. There are 3 major clinical causes of enlarged facial pores, namely high sebum excretion, decreased elasticity around pores, and increased hair follicle volume. Thus, one way of reducing the effects of skin pores on skin topographic features is to decrease excessive production and accumulation of sebum.

The overproduction of sebum by sebaceous glands also plays a role in hair care. Excessive sebum production of the sebaceous glands of the scalp is the cause of greasy hair, which is considered a significant aesthetic problem. Many cosmetic treatments, in the form of medicated shampoos and lotions, are proposed to calm the scalp's overproduction of sebum. However, cosmetics companies continuously seek new products, especially if obtained from natural ingredients. The seborrhea is involved in the occurrence of dandruff, a disorder of the scalp characterized by patches of abundant and loosely adherent flakes, usually accompanied by itching. Dandruff affects 50% of the world population. This accentuated desquamation of the scalp can evolve into seborrheic dermatitis, which is a severe form of dandruff accompanied by inflammation and erythema. The etiology of dandruff and seborrheic dermatitis appears to be dependent upon three factors: sebaceous gland secretions, micro-flora (lipophilic fungi Malassezia particularly M. globosa and M. restricta) metabolism, and individual susceptibility. The regulation of sebum production (is therefore a pivotal issue for the prevention of dandruff and seborrheic dermatitis, and the present invention is related with this problem, among others.

Finally, compounds suitable to regulate sebum production can also find application in products for intimate hygiene, since the female external genitals have many sebaceous glands. Mons pubis, labia majora, labia minora and the external side of the vaginal vestibule are rich in sebaceous glands and their sebum secretion interacts with the bacterial microflora, regulating the pH of the genital area. The fresh sebum does not contain significant quantities of free fatty acids, but these are released as an effect of the lipases produced by bacteria, inducing the acidification of the genital environment. The regulation of sebum can therefore represent an important condition for preventing alterations of the genital microflora, irritations, itching, etc.

As a consequence, the industry is strongly interested in finding new agents suitable to reduce sebum production. Ideally the agents are of natural in origin, easy to produce, readily storable, safe and usable in many different preparations, particularly in cosmetic and dermatological preparations for skin and hair care and in preparations for intimate hygiene.

The dermatologic literature contains research citing a number of substances that have been investigated for their ability to reduce sebum, such as e.g. retinoids like 13-cis retinoic acid (isotretinoin), all-trans-retinoic acid, adapalene, their salts or derivatives, androgen inhibitors like spironolactone and cyproterone, antibiotics, preferably clindamycin, erythromycin and tetracycline, and antiandrogens. However, these are mostly prescription medications intended primarily to treat acne with a secondary intent of reducing sebum. Other known sebum reducing agents comprise e.g. niacinamide, 5alpha-reductase inhibitors D-panthenol, alpha-hydroxy acids, such as e.g. salicylic acid and lactic acid, pyruvic (alfa-keto acid) acids, aliphatic dicarboxylic acids, such as e.g. azelaic acid, L-carnitine, bakuchiol, 1,2-decanediol, senkyunolide-A and senkyunolide-A containing Apium graveolens seed oil, Quillaja saponaria extract, Enantia chlorantha bark extract, Spiraea ulmaria extract, butyl avocate, vitamin B6 (also known as pyridoxine) or its salts, vitamin B3 (also known as niacin or nicotinic acid) or its salts or derivatives, benzoylperoxide, phloretin, Camellia sinensis extract and contained polyphenols such as e.g. epigallocatechin-3-gallate, red clover (Trifolium pretense) flower extract, soybean (Glycine soja) seed extract, isoflavonoids or isoflavonoid containing extracts, preferably biochanin A, genistein, daidzein, genistin, and daizin.

Barrier/Mechanical, Adherens and Tight Junctions/Differentiation:

The body surface of terrestrial animals and humans is exposed to air and to mechanical stress, both incompatible with the persistence of living cells at the direct interface between an organism and its environment. The stratifying epidermis of the skin physically separates the organism from its environment and serves as its first line of structural and functional defense against dehydration, chemical substances, physical insults and micro-organisms. The living cell layers of the epidermis are crucial in the formation and maintenance of the barrier on two different levels. First, keratinocytes ultimately form the outermost protective dead layer of the skin through a complex spatial and temporal differentiation process. Impairment of this differentiation results in a reduced stratum corneum (SC) barrier function, as can be seen e.g. in atopic dermatitis. Second, the living cell layers themselves form a barrier by providing tight mechanical cohesion between the cells of the same and different epidermal layers. To establish this barrier the viable cells have to connect to each other by intercellular junctions that link intercellular contacts to the cytoskeleton, such as tight junctions, (corneo) desmosomes and adherens junctions.

Adherens junctions are intercellular structures that couple intercellular adhesion to the cytoskeleton thereby creating a transcellular network that coordinate the behavior of a population of cells. Adherens junctions are dynamic entities and also function as signal platforms that regulate cytoskeletal dynamics and cell polarity. As such, they regulate a diverse range of other cellular processes next to adhesion, such as cell shape, division, growth, apoptosis and barrier function. The molecular basis of adherens junctions is formed by two cell adhesion receptor complexes, the classical cadherin/catenin complex and the nectin/afadin complex, which both can link to the actin cytoskeleton. Classical cadherins are single transmembrane Ca²⁺-dependent cell adhesion molecules that at their cytoplasmic face interact with catenins. Two types of classical cadherins are expressed in the epidermis: P-cadherin (cadherin 3), expressed in the basal layer mainly around and in hair follicles, and E-cadherin (cadherin 1) found in all layers of the epidermis.

Desmosomes are “mechanical” junctions, involved primarily in cell cohesion [14]. They are composed of the desmosomal cadherins, which, similar to the classical cadherins of adherens junctions, are part of the cadherin superfamily. Desmogleins 1-4 and desmocollins 1-3 are found in the human epidermis. The intracellular ends of desmosomal cadherins are inserted in the molecular network of adaptor proteins forming desmosomal plaques, to which keratin filaments bind. As keratinocytes move through the epidermal layers, they constantly form and retrieve desmosomes at the cell periphery. During this turnover, the molecules that compose junctions (even without physical dissociation of the structure) are also constantly replaced. According to the level of keratinocyte differentiation, desmogleins 2 and 3 from the lower epidermal compartment are progressively substituted by desmogleins 1 and 4 in the upper viable epidermal layers. In the same way desmocollin 3 is replaced by desmocollin 1. This differentiation-dependent composition of desmosomes coincides with the increase of their mechanical stability.

Tight Junctions (TJ) are occluding junctions. They seal the intercellular spaces between epithelial cells and the ‘tightness’ of this structure is dynamically regulated by environmental factors and addresses physiologic needs. A number of TJ proteins have been identified in human (and/or murine) epidermis and their cultured keratinocytes. They include claudins, TJ associated MARVEL protein (TAMP) and junctional adhesion molecule (JAM) transmembrane families as well as several TJ plaque proteins (e.g. Zonula occludens/ZO and cingulin). Interestingly, most of the TJ proteins, identified by immunostaining of the epidermis, are localized to the cell-cell borders of the stratum granulosum (e.g., cldns-1, -4, -6, -7, -11, -12, -18, occludin, ZO-1, ZO-2, cingulin), where the functional TJ barrier has been found. Functional evidence that epidermal barrier function requires a tight junction component came from claudin-1 deficient mice, which die of massive transepidermal water loss (TEWL) due to impaired barrier function of the stratum granulosum. Using cultured keratinocytes, it was shown that, TJs form a barrier to water and molecules of different size as well as ions.

Agents that reinforce epidermal integrity by stimulating junctional genes and proteins and thereby increase defense functions also promote scalp homoeostasis and therefore can be expected to be also beneficial for dandruff, especially if these agents also possess sebum reducing activity.

Sebum/Barrier Pollution:

According to the annual report of the World Health Organization (WHO) titled State of Global Air 2017, over 90% of the world's population live in areas with unhealthy air. The term air pollution includes but is not limited to the exhausts of traffic, not to forget the exhaust of industry in this context. Air pollution is meaning the released gas pollutants but also the released particles in this context. But also the particles by abrasion of rubber wheels are included. The particles which are involved in air pollution might have bound polycyclic aromatic hydrocarbons (PAH) but are not limited to these PAH rich ones. Also carbon black particles released by printers are an air pollution problem which is occurring indoor. Another problem is the generation of air pollution by indoor heating and cooking with coal or firewood.

One of the most common components of air pollution is particulate matter (PM), which is classified as PM10, fine PM, and ultrafine particles according to the particles' aerodynamic diameter. PM10 (particles of less than 10 μm diameter) is composed of particles from dust, industrial emissions, and traffic emissions. A smaller PM diameter with less than 2.5 μm is defined as fine PM (PM2.5); PM2.5 is primarily comprised of organic carbon compounds, nitrates, and sulfates. Epidemiological investigations into contamination, especially ambient air pollution, indicated that the PM is correlative with the progression of inflammatory skin diseases such as atopic dermatitis, acne, psoriasis, and allergic reactions.

Environmental/exogenous factors are implicated in the quality and quantity of secreted sebum. Thus, e.g. pollution: whether natural or due to human activity (ozone, industry, farming, smoking, etc.), affects the composition of sebum and is also thought to stimulate its secretion. Thus, low concentrations (1 and 10 μg/ml) of PM2.5 were shown to promote lipid synthesis in cultured sebocytes (Q. Liu et al., Int J Mol Med. 2017, 40(4), 1029-1036).

Furthermore, a study on a human keratinocyte cell line demonstrated interactions between COX2/PGE2 (Prostaglandin E2) and filaggrin in the development of skin barrier dysfunction providing evidence that PMs trigger significant and dose-dependent increases in COX2 protein levels, mRNA expression, promoter activity and PGE2 production, ultimately resulting in filaggrin down-regulation.

The exposure of human skin to repeated air pollution was also shown to support formation of pigment spots due to a crosstalk of keratinocytes and melanocytes, the melanin producing cells of the skin.

Therefore, an agent which on the one hand decreases sebum production of the sebaceous glands and on the other hand strengthens the epidermal integrity by stimulating desmosomal, adherens and tight junction proteins and thereby reduces the penetration of particulate matter and other lipid synthesis promoting environmental stimuli is especially advantageous.

Antimicrobial Peptides and Proteins

Antimicrobial peptides or proteins (AMPs) represent an ancient and efficient innate defense mechanism which protects interfaces from infection with pathogenic microorganisms. In human skin AMPs are produced mainly by keratinocytes, neutrophils, sebocytes or sweat glands and are either expressed constitutively or after an inflammatory stimulus. Skin lesions of patients with atopic dermatitis show a diminished expression of the beta-defensins and the cathelicidin LL-37. In addition, decreased levels of AMPs are associated with burns and chronic wounds. In contrast, overexpression of AMPs can lead to increased protection against skin infections as seen in patients with psoriasis and rosacea, inflammatory skin-diseases which rarely result in superinfection. In other skin diseases, e.g. in patients with acne vulgaris, increased levels of AMPs are often found in inflamed or infected skin areas indicating a role of these peptides in the protection from infection. The broad spectrum of antimicrobial activity, the low incidence of bacterial resistance and their function as immunomodulatory agents are attractive features of AMPs for their clinical use.

Defensins, comprising the alpha and beta families, are one of the largest and most-studied families of AMPs in mammals. Human defensins have a broad spectrum of antimicrobial activity against gram-positive and gram-negative bacteria, viruses, fungi, and some protozoa and are important components of the innate immune system.

Beta defensins are cationic peptides with antimicrobial activity that defend epithelial surfaces including the skin, gastrointestinal, urinary and respiratory tracts. Human β-defensin 1 peptide (hBD-1), encoded by the DEFB1 locus and acts against gram-positive and negative bacteria. After reduction of disulphide-bridges, hBD-1 becomes a potent AMP against the opportunistic pathogenic fungus Candida albicans. It shows synergistic effect with LL-37 or lysozyme against S. aureus and E. coli.

S100 proteins are low molecular weight cationic proteins characterized by two calcium-binding EF-hand motifs. They are involved in a variety of cellular processes such as calcium-dependent cell signaling, cell growth, and antimicrobial defense.

Psoriasin (S100A7), a Ca²⁺ binding S100 protein, was discovered in psoriatic lesions and is expressed at low levels in normal epithelial cells. The focal expression of psoriasin is found in the skin, especially in localizations associated with high density of bacteria. In addition, the peptide accumulates in the epidermis of sebaceous skin as well as sebaceous glands and is secreted to the external skin surface. It exhibits an antibacterial activity preferentially against E. coli. Another member of the S100A family with an antimicrobial activity is calprotectin, a heterocomplex of the two calcium-binding proteins S100A8 and S100A9. Calprotectin exerts antibacterial properties inter alia against E. coli, Klebsiella spp., Staphylococcus aureus and S. epidermidis, as well as fungistatic activity toward the fungus C. albicans.

Adrenomedullin (AM) is a multifunctional peptide produced by a wide variety of cells, including keratinocytes. It has a role as a growth regulatory factor of the skin and contributes as an antimicrobial agent in the integument's protective barrier.

Epidermal Integrity/Inflammation/Post-Inflammatory Hyperpigmentation:

Skin insults that result in inflammation can not only induce sebum production but can also result in postinflammatory hyperpigmentation (PIH), particularly in people with darker skin (skin type III to VI). Among such insults are e.g. acne lesions. Also, air pollution/particulate matter (PM) emerge as etiologic agent for the development of melasma and other facial pigmentary dyschromias.

PGE2 is one of the most abundant metabolites of arachidonic acid, generated through an enzymatic cascade controlled by cyclooxgenase (COX) enzymes. COX-2 is induced in response to multiple inflammatory stimuli in skin cells. PGE2 mediates its effects in melanocytes through the G-protein coupled receptors EP1 and EP3 resulting in activation of PKC-ζ (protein kinase C zeta). PGE2 stimulates melanocyte dendrite formation and melanosomes transfer. Furthermore, COX-2 knock-down in melanocytes was shown to decrease the expressions of tyrosinase, TRP-1, TRP-2, gp100 and MITF and also reduced tyrosinase enzyme activity. Additionally, COX-2 siRNA-transfected melanocytes showed markedly reduced alpha-melanocyte stimulating hormone (α-MSH)-induced melanin production.

COX-2 and PGE2 thus were proven to play an important role in PIH.

The prior art related to the microalgae exploitation in the field of cosmetics and dermatology offers some examples:

FR 2980698 A1 (Gelyma) discloses the modulation of sebum production by exploiting the combined activity of extracts from the microalgae Tetraselmis chui and the macroalgae Fucus spiralis. Tetraselmis chui is obtained by biotechnology with controlled metabolic induction cultivation process in order to induce mineral bioaccumulation, presently bio-available zinc. Thus, the extract is prepared from Tetraselmis chui obtained by culture in medium enriched in zinc and is characterized by a zinc content of 10 to 2000 ppm. According to the disclosure Tetraselmis chui differs from Tetraselmis suecica by its chemical composition. Sebum reducing activity due to 5alpha-reductase inhibition as well as anti-inflammatory efficacy by interleukin (IL)-8 and tumor necrosis factor (TNF)-alpha inhibition is only shown for the combined extracts of Tetraselmis chui and Fucus spiralis, not for the individual extracts so that it is not clear if only one or both of them exhibit these activities.

KR2013015037 A discloses an extraction method for isolating substances with anti-inflammatory and anti-acne functions from Tetraselmis suecica or Chlorella ellipsoidea. The effective extract is obtained by desalting and dewatering the microalgae biomass and processing it into powder. After this pre-treatment the microalgae is either directly extracted with ethyl acetate (TS-6000) or is extracted with 1% sodium hydroxide solution (=alkaline extraction). The alkaline mixture is then neutralized with HCl, the extract solution is separated by filtration, ethyl acetate is added and the organic phase is separated (TS-2000). These 2 lipophilic extracts inhibit the secretion of inflammatory cytokines of macrophages, namely nitric oxide (NO), IL-6 and TNF-alpha. The anti-acne effect of the extracts is deduced from anti-bacterial activity against Propionibacterium acnes. There is no indication of Tetraselmis suecica extracts having sebum reducing activity.

WO2016020339 A2 (Cutech) discloses extracts of microalgae, halophytes and psammophilous plants, obtainable by extraction with a solvent selected from the group consisting of C1-C4 aliphatic alcohols, ethyl acetate, water or their mixtures, showing activity as regulators of the metabolism of human sebaceous glands. Explicitely described is the sebum reducing activity of extracts obtained from microalgae belonging to the genus Chlorococcum, Thalassiosira, Monodus and Chaetoceros. Extracts from Tetraselmis are not disclosed.

FR 2894473 A1 (Daniel Jouvance) discloses the use of preparations obtained from some microalgae biomass paste or suspension (Chromulina, Asterionella and Tetraselmis) for inhibiting the enzymes involved in the metabolism of fatty acids and lipids namely acetylcoenzyme A carboxylase (ACC), phosphodiesterase (PDE), glyceraldehyde 3-phosphate deshydrogenase (Ga3PDH), fatty acid synthase (FAS), lipoprotein lipase (LPL) in adipocytes or pre-adipocytes. No biological data are given.

C. Sansone et al. (Scientific Reports (2017), 7, 41215 CODEN: SRCEC3; ISSN: 2045-2322) describe an ethanol/water extract of Tetraselmis suecica containing high levels of carotenoids such as the xanthophylls lutein, violaxanthin, neoxanthin, antheraxanthin and loroxanthin esters and its strong antioxidant and prostaglandin E2 (PGE2) levels reducing activity in human lung cancer cells (A549) damaged by H₂O₂.

US2010143267 A1 (Symrise) describes the use of extracts obtained from Tetraselmis sp. amongst others for stimulating the level of cornified envelope protein components such as filaggrin and/or involucrin. Extracts are obtained by extracting viable, freeze-dried or dried cells of Tetraselmis sp., preferably Tetraselmis suecica, with a liquid extractant selected from the group consisting of hexane, ethyl acetate, ethanol, water, methanol, isopropanol and mixtures of two or more of these extractants for up to 24 h at a temperature of not more than 50° C. According to Examples 33-40 and 41-48, the sequential ethanol extract at 5 μg/ml is the most effective extract for increasing involucrin and also filaggrin in ex vivo human skin.

WO2017068424 discloses cosmetic or dermatological compositions comprising dihydromyricetin and a zinc salt, preferably zinc gluconate, and advantageously biochanin A or a plant extract comprising biochanin A, for the treatment of acne and acne-prone oily skin. The composition can also contain a polyol, preferably chosen from the group of xylitol, sorbitol or mannitol. There is no function given for the polyol in this composition and no indication that the polyol possesses sebum reducing activity by itself.

The same is true for EP 2583662 which discloses a composition comprising a meroterpen to manage oily skin with tendency to develop acne.

WO2017120468 describes the therapeutic use of nalbuphine for treatment of pruritic condition comprising e.g. atopic dermatitis, seborrheic dermatitis, eczema, acne vulgaris, or visceral diseases complicated with pruritus with mannitol being given as part of a delivery system for sustained release including about 0.5% to about 80% locust bean gum, about 5% to about 80% xanthan gum, about 20% to about 80% mannitol and about 0.5% to 80% calcium sulfate dehydrate. There is no indication given on sebum reducing activity of mannitol.

The biodiversity of microalgae is very high and in great part still uncertain: to date about 35,000 species of microalgae have been described but the number of unknown species is estimated to vary from 200,000 to 800,000. The adaptability of these organisms allows them to synthesize rare and biologically active compounds suitable to sustain specific and diversified environmental stresses or to compete successfully with other organisms. It is generally known that different biological species comprise different substances. Thus, effects obtainable by use of one microalgal species cannot be used to predict the effects obtainable by use of a different microalgal species.

The problem of the present invention was, therefore, to provide new agents suitable to reduce sebum production and a method to obtain the new agents.

Another problem, to be solved by the present invention, was to obtain new cosmetic or dermatological compositions and products for treating or preventing dysfunctions of the human hair and/or skin and the use of these compositions for cosmetic and therapeutic applications.

The problems relating to the present invention are solved by the following:

A Tetraselmis suecica extract, wherein the Tetraselmis suecica extract comprises total minerals at ≥10 wt. % of the total composition;

wherein the Tetraselmis suecica extract comprises mannitol at ≥5 wt. % of the total composition;
wherein the Tetraselmis suecica extract comprises total galactose, which is the sum of free and bound galactose, at ≥3 wt. % of the total composition;
wherein the Tetraselmis suecica extract comprises total glucose, which is the sum of free and bound glucose, at ≥2 wt. % of the total composition;
wherein the Tetraselmis suecica extract comprises total amino acids at ≥3 wt. % of the total composition;
wherein the Tetraselmis suecica extract comprises total nitrogen at ≥2 wt. % of the total composition. The component proportions are based on the dried extract weight.

A method of obtaining a Tetraselmis extract as well as the product of said method; the method of obtaining a Tetraselmis extract comprising the step of extracting, (preferably viable), freeze-dried or dried cells of Tetraselmis, with a liquid extractant selected from the group consisting of 2-propanone, ethanol, water, methanol, isopropanol and mixtures of two or more of these extractants, and wherein the extraction comprises: a) exposition of the cell material to the extractant for up to 8 h at a temperature higher than 60° C. and b) removal of the cell material to obtain the extract. Preferably the extract is a dried Tetraselmis suecica extract; in this case the method comprises additionally the step: c) removing the extracting extractants. It is favorable that the extraction step is performed on viable, freeze-dried or dried cells of Tetraselmis.

A combination composition comprising a Tetraselmis extract and further comprising niacinamide.

A Tetraselmis extract concentrate, wherein the Tetraselmis extract concentrate comprises 0.5 to 80 wt. % Tetraselmis extract or combination composition of a Tetraselmis extract and niacinamide, wherein the Tetraselmis extract concentrate further comprises 0.5 to 90 wt. % water; wherein the Tetraselmis extract concentrate further comprises 0.5 to 90 wt. % carrier; wherein the Tetraselmis extract concentrate further comprises 0.1 to 5 wt. % of one or more preservative or preservative system.

Tetraselmis suecica algae have been cultured in Italy for some time, e.g. cultured by an Italian hatchery in Orbetello. Furthermore, six strains of Tetraselmis suecica of different origin are available from CCAP (Culture Collection of Algae and Protozoa), e.g. CCAP 66/4, CCAP 66/22A, CCAP 66/22B, CCAP 66/22C, CCAP 66/22D and CCAP 66/38. However other sources, such as culture collections of Tetraselmis suecica algae can be considered as a potential source of biological material for the present invention.

Tetraselmis biomass can be obtained by cultivation in photobioreactors or in large polyethylene bags or tanks, under daylight or artificial light. The cultivation can occur indoors or outdoors. When the microalgal biomass reaches a suitable cell density, it can be harvested by centrifugation or sedimentation or flocculation or with other techniques suitable to preserve the integrity of the cell material. The harvested biomass is then used fresh (viable) or dried e.g. by freeze- or spray-drying or processed by other suitable technique. As raw material for the extraction, so far unextracted biomass or residual biomass resulting from a prior extraction or processing with organic solvents such as e.g. ethyl acetate, hexane, cyclohexane, acetone, carbon dioxide, methanol, ethanol, propanol, iso-propanol, 1-butanol, 2-butanol, tert-butanol or a mixture of organic solvents can be used.

In particular, the present invention relates to a novel method of obtaining a Tetraselmis extract. More specifically, this process removes coloured components in the Tetraselmis extract. This has the effect of increasing the lightness in the extract.

Surprisingly, it was now found that extracts of the microalgae Tetraselmis suecica highly efficiently reduce sebum production.

Furthermore, it was surprisingly found that Tetraselmis extracts potently upregulate many genes involved in epidermal junctions, such as desmosomal (“mechanical”), tight, adherens and gap junctions relevant for cell-to-cell adhesion and tissue integrity as well as allowing of the exchange of ions, second messengers, and small metabolites between adjacent cells.

Furthermore, Tetraselmis extract surprisingly modulates genes relevant for differentiation and re-epithelialization relevant for processes such as wound healing, tissue regeneration and barrier formation.

Additionally, Tetraselmis extract surprisingly increased the gene expression of antimicrobial peptides.

Astonishingly, the expression of aquaporin 3 which is important for water/glycerol-transport in the skin, was also stimulated by treatment with Tetraselmis extract.

Additionally, Tetraselmis extract surprisingly was discovered to potently down-regulate COX-2 gene expression as well as inhibit COX-2 enzyme activity which not only results in reduced sebum production and inhibition of inflammatory processes and erythema but can also be expected to have a beneficial effect on PIH of human skin.

Therefore, in a first aspect, the invention relates to a Tetraselmis suecica extract comprising:

a) total minerals more than or equal to 10 wt. % of the total composition,
b) mannitol more than or equal to 5 wt. % of the total composition,
c) total galactose, which is the sum of free and bound galactose, more than or equal to 3 wt. % of the total composition,
d) total glucose, which is the sum of free and bound glucose, more than or equal to 4 wt. % of the total composition,
e) total amino acids more than or equal to 3 wt. % of the total composition, and
f) total nitrogen more than or equal to 2 wt. % of the total composition.
The component proportions are based on the dried extract weight.

The Tetraselmis suecica extract is thus distinguished over the prior art in its composition. In particular, the results of the extraction at low temperature as done in document EP 2 193 785 A2 are shown in comparison to the present high temperature extraction in Table 2. This comparison shows striking differences in sugar and amino acid distribution. Notably, the glucose level is higher for the high temperature extraction, leading to a glucose amount of more than 4 wt. % of the total composition, compared to only 3.5 wt. % in the state of the art.

The Tetraselmis suecica extract is preferably obtained by extracting cells of Tetraselmis suecica with a liquid extractant at a temperature higher than 60° C. The Tetraselmis suecica cells are preferably used either fresh (viable), dried, e.g. by freeze- or spray-drying, or processed by other suitable techniques.

The liquid extractant suitable for extraction is a polar solvent, i.e. a solvent with a dielectric constant greater than 15. Preferably, the extraction of the Tetraselmis suecica cells is carried out with a polar solvent selected from the group consisting of 2-propanone, ethanol, water, methanol, isopropanol and mixtures of two or more of these solvents.

The extraction is carried out by exposing the cell material to the extractant for up to 8 h at a temperature higher than 60° C. After extraction of the Tetraselmis suecica cells is completed, the cell material is removed to obtain the extract. Preferably the extract is a dried Tetraselmis suecica extract. In this this case the extracting extractants are removed from the extracted substances.

Hereby, an exposition time of 0.5 to 4 h is preferred and provides a Tetraselmis extract capable of significantly reducing sebum production of the skin. Even more preferred is an exposition time of the cell material to the extractant of 1 to 3 hours which offers an extract with increased capabilities of reducing sebum (see operational Example 3 and 6).

The resultant extract also does not show an intensive dark green color, but a beige color which is preferred when applying the gained Tetraselmis extract in medical and/or cosmetic and/or other compositions (see operational Example 1).

Furthermore, a temperature of more than or equal to 70° C. is preferred. This temperature was found to influence the sebum reduction capabilities of the obtained Tetraselmis extract beneficially, but also provided the preferred coloration of the Tetraselmis extract.

Even more preferred is a temperature during exposition of more than or equal to 75° C., most preferred in the range of 75 to 95° C. This Temperature not only provides the above-named benefits of coloration and sebum reducing capabilities, but also provides a special Tetraselmis extract which surprisingly influences the gene expression of genes involved in epidermal junctions, antimicrobial peptides, water/glycerol-transport in the skin as well as COX-2 regulation (see operational Examples 1, 5, 7, 8, 9).

Preferably, the Tetraselmis suecica extract has a total galactose content, which is the sum of free and bound galactose, of 6 to 12 wt. % of the total composition, even more preferably between 8 to 11 wt. % of the total composition, based on the extract dry weight. This also leads to improved skin hydration properties of cosmetics and medications based on the Tetraselmis suecica extract.

Preferably, the Tetraselmis suecica extract has a total glucose content, which is the sum of free and bound glucose, of 4 to 10 wt. % of the total composition, even more preferably between 6 to 9 wt. % of the total composition, based on the extract dry weight. This also leads to improved skin hydration properties, especially in cosmetics and medications based on the Tetraselmis suecica extract.

Preferably, the Tetraselmis suecica extract has a total Arginine content, which is the sum of free and bound Arginine, of 0.2 to 1.5 wt. % of the total composition, even more preferably between 0.6 to 1 wt. % of the total composition, based on the extract dry weight.

Preferably, the Tetraselmis suecica extract has a total Asparagine content, which is the sum of free and bound Asparagine, of 0.2 to 1.0 wt. % of the total composition, even more preferably between 0.3 to 0.5 wt. % of the total composition, based on the extract dry weight.

Preferably, the Tetraselmis suecica extract has a total Aspartic acid content, which is the sum of free and bound Aspartic acid, of less than 0.7 wt. % of the total composition, even more preferably between 0.2 to 0.3 wt. % of the total composition, based on the extract dry weight.

Preferably, the Tetraselmis suecica extract has a total Ornithine content, which is the sum of free and bound Ornithine, of less than 1.0 wt. % of the total composition, even more preferably between 0.4 to 0.6 wt. % of the total composition, based on the extract dry weight.

In the present application, as indicated above and throughout the application, a Tetraselmis suecica extract is preferably a dried Tetraselmis suecica extract, obtained by removing the extracting extractants, either partially or preferably completely. If the extractants are removed partially, then the remaining extractants are present in the extract in an amount of between 0.5 to 10 wt. %. In some cases, it is preferred to employ the Tetraselmis suecica extract in its liquid native form, without the drying step. Alternatively, further substances may be added before partial drying, such as glycerin. In such cases, typically and aqueous glycerin solvent system is achieved, with the active components dissolved therein.

This extract was found to be highly efficient in reducing sebum production. This was particularly effective for extracts comprising mannitol in 10 to 14 wt. %. This is backed by operational Examples 3 and 6 describing the sebum reducing effect of such an extract. Preferably, the extract comprised total minerals of 15 to 30 wt. %. It is also preferred for the extract to comprise 7 to 20 wt. % total galactose. An amount of galactose within the preferred range hereby increases shelf life of the extract. Furthermore, it is preferred for the extract to contain 5 to 13 wt. % total glucose, which is also increasing shelf life of the extract. Additionally, it is also preferred for the extract to contain at least 6 wt. %, but no more than 16 wt. % total amino acids. Finally, it is preferred for the extract to contain total nitrogen of 3 to 7 wt. % percent of the total composition.

As indicated above, the extract may be in dried form, and the above components are calculated based on the dried extract, although this can also be employed in liquid form, such as a non-dried native extract.

In a preferred first variation of the first aspect, the Tetraselmis suecica extract comprises:

a) total minerals 11 to 25 wt. % of the total composition,
b) mannitol 6 to 15 wt. % of the total composition,
c) total galactose, which is the sum of free and bound galactose, 4 to 15 wt. % of the total composition,
d) total glucose, which is the sum of free and bound glucose, 4 to 10 wt. % of the total composition,
e) total amino acids 4 to 10 wt. % of the total composition, and
f) total nitrogen 3 to 5 wt. % of the total composition.
Again, the component proportions are based on the dried extract.

The extract according to the present invention is characterized by a higher galactose and glucose content. In addition, of the amino acids Arginine and Asparagine are enriched compared to a Tetraselmis suecica extract obtained by extraction at room temperature (see Table 2). Increased Arginine and Asparagine are assumed to increase epidermal skin hydration by their water-holding capacity, despite sebum reducing effect of the extract. Galactose and glucose, as well as Asparagin, also increase the shelf life of the extract.

A Tetraselmis suecica extract according to the first variation of the first aspect hereby proves to have an especially pronounced sebum reducing effect.

In a second aspect, the invention relates to a method of obtaining a Tetraselmis extract comprising the step of extracting viable, freeze-dried or dried cells of Tetraselmis, with a liquid extractant selected from the group consisting of 2-propanone, ethanol, water, methanol, isopropanol and mixtures of two or more of these extractants, and wherein the extraction comprises: a) exposition of the cell material to the extractant for up to 8 h at a temperature higher than 60° C. and b) removal of the cell material to obtain the extract. Preferably the extract is a dried Tetraselmis suecica extract; in this case the method comprises additionally the step c) removing the extracting extractants.

Hereby, an exposition time of 0.5 to 4 h is preferred, as this timespan not only shortens the production time of the Tetraselmis extract and therefore reducing production cost and effort, but also provides a Tetraselmis extract capable of significantly reducing sebum production of the skin.

Even more preferred is an exposition time of the cell material to the extractant of 1 to 3 hours. An exposition time within this range hereby further decreases production time and production cost, but also offers an extract with increased capabilities of reducing sebum (see operational Example 3 and 6) which also does not show an intensive dark green color, but a beige color which is preferred when applying the gained Tetraselmis extract in medical and/or cosmetic and/or other compositions.

Furthermore, a temperature of more than or equal to 70° C. is preferred. This temperature was found to influence the sebum reduction capabilities of the obtained Tetraselmis extract beneficially, but also provided the preferred coloration of the Tetraselmis extract.

Even more preferred is a temperature during exposition of more than or equal to 75° C., most preferred in the range of 75 to 95° C. This Temperature not only provides the above-named benefits of coloration and sebum reducing capabilities, but also provides a special Tetraselmis extract which surprisingly influences the gene expression of genes involved in epidermal junctions, antimicrobial peptides, water/glycerol-transport in the skin as well as COX-2 regulation (see operational Examples 1, 5, 7, 8, 9).

The ratio of extractant to Tetraselmis matrix is preferably between 80:1 and 3:1. More preferably 20:1 to 8:1. This relatively low ratio with less extractant leads to an improved decoloration effect.

Particularly preferred general extraction processes are maceration, re-maceration, digestion, agitation maceration, vortex extraction, ultrasonic extraction, counter current extraction, percolation, re-percolation, evacolation (extraction under reduced pressure), subcritical or supercritical fluid extraction, diacolation and solid/liquid extraction under continuous reflux. Percolation is even more preferred and was found to have advantageous upscaling properties.

A preferred size reduction method is freeze grinding.

Preferred solvents for the extraction process are water or mixtures of organic solvents, e.g. methanol, ethanol, isopropyl alcohol, acetone with water. Preferably hot water with a temperature above 60° C., and more particularly above 70° C. is used.

Another preferred method for removing the extracting extractants is by adding glycerin to the aqueous extract solution after removal of Tetraselmis biomass/cells and removing part of the water. Further preferred is then adding a preservative or preservative system such as potassium sorbate, sodium benzoate and lactic acid to the extract.

Optionally, the extraction times can be modified depending on the starting material, the extraction process, the extraction temperature, and the ratio of solvent to raw material.

After the extraction process, the crude extracts obtained may optionally be subjected to other typical steps, such as, for example, purification and/or further decoloration.

In a third aspect, the Tetraselmis extract according to the first aspect is obtained by the method of the invention according to the second method aspect as described above and its preferred variants.

By applying the inventive method with a higher extraction temperature, it was possible to obtain Tetraselmis extracts with favorable properties, especially compositions, which were effective in ameliorating skin conditions, diseases or blemishes.

Furthermore, a composition acquired by the method described in the previous aspect of the invention can beneficially influence tight junction dynamics (operational Example 12) and is especially suitable for influencing the gene expression of genes involved in epidermal junctions, antimicrobial peptides, water/glycerol-transport in the human skin as well as COX-2 regulation (see operational Examples 1, 5, 7, 8, 9)

It is especially preferred in a method or product as described above that the Tetraselmis classification is Tetraselmis sp., more preferably Tetraselmis suecica. The observed skin treatment or protection advantages are particularly pronounced for Tetraselmis suecica extracts, although Tetraselmis in general is suitable.

Synergism with Niacinamide:

Niacinamide (I), also known as nicotinamide, is a water-soluble vitamin in the vitamin B family, specifically the vitamin B3 complex and is found in food, used as a dietary supplement, and cosmetic ingredient in skin and hair care.

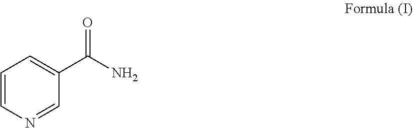

It is a known sebum reducing (Z. D. Draelos et al., J Cosmet Laser Ther. 2006, 8(2), 96-101), potent anti-inflammatory and anti-acne agent (F. M. Walocko et al., Dermatol Ther. 2017, 30(5). doi: 10.1111/dth.12481). Nicotinamide also improves the epidermal permeability barrier in vivo.

According to the invention, a further fourth aspect of the invention is a combination composition, comprising the Tetraselmis extract according to the invention described herein, further comprising niacinamide.

It was not foreseeable that Tetraselmis extracts in combination with niacinamide exhibit particularly good sebum reducing activity. Surprisingly, it was found by our experimentation that Tetraselmis extract and niacinamide highly synergistically reduce the total lipids content of sebaceous glands, i.e. sebum level. This is backed by operational Example 4 of the present invention. The enhancing effect of Tetraselmis on Niacinamide is unexpected.

Particularly effective were combinations in the composition, wherein the weight ratio range of Tetraselmis extract to niacinamide is 1:10000 to 1:1, preferably 1:2500 to 1:1, more preferably from 1:500 to 1:10, most preferably 1:400 to 1:300. Again, the weight ratios are calculated based on Tetraselmis extract dry weight.

Preferred is a sebum reducing composition, consisting of or comprising Tetraselmis extract and niacinamide, wherein the Tetraselmis extract is used in an amount of 0.01-0.05 wt. % extract and niacinamide in amount of for 0.5 to 5 wt. %, based on the total weight of the final (skin care) product.

It was found that the amounts of Tetraselmis extract and niacinamide in the formulation adjusted in this way have synergistically sebum reducing capabilities.

Furthermore, the Tetraselmis extract can be used in form of an extract concentrate. Preferably, according to a fifth invention aspect said Tetraselmis extract concentrate comprises:

a) 0.5 to 80 wt. % Tetraselmis extract according to the first aspect or a combination composition as described above according to the invention,
b) 0.5 to 90 wt. % water,
c) 0.5 to 90 wt. % carrier.
The weight ratios are calculated based on Tetraselmis extract dry weight.

More preferred is a content of 0.5 to 30 wt. % Tetraselmis extract or combination composition as described above. Furthermore, a content of 10 to 80 wt. % water is more preferably employed. Additionally, a content of 15 to 70 wt. % carrier is preferred.

Preferably, the above concentrate further comprises 0.1 to 5 wt. % of one or more preservative or a preservative system. In another preferred form, the concentrate comprises also stabilizers.

Even more preferred is the use of 0.5 to 2 wt. % of one or more preservatives or preservative systems or stabilizers, as this amount of preservatives or preservative systems or stabilizers was found to positively influence the shelf life of the extract concentrate as described in the fifth aspect without negatively affecting any positive features, such as sebum-removal-capabilities of the prepared Tetraselmis extract concentrate.

The amount of the respective components is chosen so that it complies with the Cosmetics Directive 76/768/EEC and EU Directive 95/17/EC. Preferably the preservatives are employed according to the classes and compounds listed in the Appendix 6, Parts A and B of the Cosmetics Directive 76/768/EEC. More specific preferable preservatives are benzoic acid, sodium benzoate, sorbic acid, lactic acid, potassium sorbate, phenoxyethanol, or combinations thereof. Lactic acid is preferred. Most preferred is sorbic acid. Preservative boosters are preferably hydroxyacetophenone, 1,2-pentanediol, 1,2-hexanediol, 1,2-octanediol or combinations thereof. However, 1,2-pentanediol may also be used in higher amounts as a secondary liquid carrier.

More preferably, the above concentrate is either a liquid or solid concentrate. If the concentrate is a liquid concentrate it advantageously comprises 1 to 70 wt. % water, more preferably 30 to 60 wt. % water.

More preferably, the Tetraselmis extract concentrate is a liquid Tetraselmis extract concentrate comprising:

a) 0.5 to 10 wt. % Tetraselmis extract or a combination composition according to the invention,
b) 1 to 70 wt. % water,
c) 0.5 to 85 wt. % liquid carrier, preferably glycerin, and
d) optionally 0.1 to 5 wt. % of a preservative or preservative system.
The weight ratios are calculated based on Tetraselmis extract dry weight.

Tetraselmis extract concentrate preferably comprises 2 to 3 wt. % Tetraselmis extract matter, preferably 2.5 wt. % Tetraselmis extract matter.

An even more preferable liquid Tetraselmis extract concentrate is one comprising the following, calculated based on dry weights:

a) 1 to 10 wt. % Tetraselmis extract or combination composition as described herein,
b) 30 to 70 wt. % water,
c) 20 to 60 wt. % glycerin,
d) optionally 0.1 to 5 wt. % of one or more preservative or preservative system.

It is particularly preferred, when the Tetraselmis extract concentrate is a liquid Tetraselmis extract concentrate comprising:

a) 0.5 to 10 wt. % Tetraselmis extract or a combination composition according to the present invention,
b) 40 to 65 wt. % water,
c) 25 to 55 wt. % glycerin,
d) 0.1 to 1 wt. % potassium sorbate
e) 0.1 to 1 wt. % sodium benzoate and
f) 0.1 to 5 wt. % lactic acid.
The weight ratios are calculated based on Tetraselmis extract dry weight.

Liquid Tetraselmis extract concentrate is preferably produced after extraction and separation of the biomass from the extract solution and by partially or complete removal of the extractant and optional addition of a liquid carrier such as e.g. glycerin, propylene glycol, butylene glycol, 1,3-propanediol, 1,2-pentanediol, 1,2-hexanediol, preferably glycerin, or mixtures of two or more of these and optional addition of a preservative or preservative system. Such systems can optionally comprise 0.1 to 5 wt. % of the preservative.

The liquid carrier 1,2-pentanediol, is particularly preferred (Hydrolite-5). It can function as a preservative, but also in combination with glycerine, 1,2-pentanediol was found to be an excellent liquid carrier combination. Preferred is that a combination of glycerine and 1,2-pentanediol as liquid carrier, together with water to form an extract concentrate.

A liquid Tetraselmis extract concentrate comprising:

a) 0.5 to 10 wt. % Tetraselmis extract or combination composition according to any one of claims 1, 2, 4 to 7, calculated based on dry weights,

b) 30 to 70 wt. % water,

c) 20 to 50 wt. % glycerin,

d) 5 to 20 wt. % 1,2-pentanediol,

e) optionally 0.1 to 5 wt. % of one or more preservative or preservative system.

Preferably the ratio of glycerin to water was from 0.3:1 to 1.2:1, while the ratio of 1,2-pentanediol to water was from 0.03:1 to 0.4:1.

A good preservative for the inventive concentrate and in particular the above system is Na-benzoate or K-sorbate, preferably in combination with lactic acid. The combination of these preservative compounds worked well with the Tetraselmis extract concentrate.

A preferred Tetraselmis extract solution can be obtained by adding glycerin to the aqueous extract solution after removal of the Tetraselmis biomass/cells and then removing part of the water. After this it is further gainful, but not necessary in all cases, to add a preservative or preservative system such as potassium sorbate, sodium benzoate and/or lactic acid to obtain a preferred solution that can be employed for the treatment of skin diseases.

A preferred Tetraselmis extract solution thus comprises 2 to 3 wt. % Tetraselmis suecica extract matter, preferably 2.5 wt. % Tetraselmis suecica extract matter, 40 to 60 wt. % water, 30 to 50 wt. % glycerin, 0.1 to 1 wt. % sodium benzoate, 0.1 to 0.5 wt. % potassium sorbate, wherein the pH adjusted to 4 to 5 with additionally comprised lactic acid.

The weight ratios are calculated based on Tetraselmis extract dry weight.

Also preferred is that the Tetraselmis extract concentrate is a solid Tetraselmis extract concentrate comprising:

a) 0.5 to 10 wt. % Tetraselmis or a combination composition according to the invention,
b) 0.5 to 8 wt. % water, and
c) 50 to 98 wt. % solid carrier, preferably maltodextrin.
The weight ratios are calculated based on Tetraselmis extract dry weight.

In another preferred form, this solid Tetraselmis extract concentrate comprises a preservative or preservative system.

The solid Tetraselmis extract concentrate is gainfully produced after extraction and separation of the biomass from the extract solution either without or with prior partially removal of the extractant and after optional addition of a solid carrier such as e.g. modified starches like maltodextrin, dextrin or cyclodextrin, lactose, modified celluloses, gums like xanthan gum, gellan gum, guar gum, gum arabic, gum ghatti, tragacanth gum or locust bean gum, silicium dioxide, preferably maltodextrin or mixtures of two or more of these by drying using suitable processes such as spray-, freeze- or vacuum drying.

The above liquid or solid Tetraselmis extract concentrates can be employed in cosmetic and/or dermatological and/or pharmaceutical products for skin and hair care and cleansing in an amount of 0.0001 to 10 wt. %, preferably 0.001 to wt. 5% and most preferably 0.005 to 3 wt. % of the final products.

It was found that these liquid or solid Tetraselmis extract concentrates show good storage properties, are easy to handle, dose and formulate.

In a further inventive sixth aspect, particularly preferred is a Tetraselmis extract or a combination composition or a Tetraselmis extract concentrate as described herein, which is used as a medicament for treating skin related diseases and medical conditions.

Especially preferred is a Tetraselmis extract as described herein, which is used as a medicament for treating or preventing dysfunctions of human hair and/or skin, seborrhoeic dermatitis (seborrhea), acne vulgaris, wound healing, tissue regeneration, post-inflammatory hyperpigmentation, inflammatory related diseases, dandruff or Pityriasis versicolor. Treatment of Pityriasis versicolor is preferably achieved by reducing Malassezia.

Hereby, it is more preferred that the Tetraselmis extract as described by the present sixth aspect, is used as a medicament for treating or preventing dysfunctions of human hair and/or skin, inflammatory related diseases, acne and dandruff, wherein it is most preferred for the Tetraselmis extract to be an extract obtained from Tetraselmis suecica according to the previously described aspects.

Interestingly, a Tetraselmis extract, preferably obtained from Tetraselmis suecica, more preferably prepared in accordance with the second inventive aspect of the present invention is especially effective when used as a medicament for preventing of treating dysfunctions of human hair and/or skin, inflammatory related diseases, acne and dandruff.

Furthermore, especially preferred is a combination composition as described by the present invention, which is used as a medicament for treating or preventing dysfunctions of human hair and/or skin, seborrhoeic dermatitis (seborrhea), acne vulgaris, wound healing, tissue regeneration, post-inflammatory hyperpigmentation, inflammatory related diseases, dandruff or Pityriasis versicolor. Treatment of Pityriasis versicolor is preferably achieved by reducing Malassezia.

Even more preferred is the use of the combination composition as described herein as a medicament for treating or preventing dysfunctions of human hair and/or skin, acne vulgaris or seborrheic dermatitis, wherein it is most preferred for the Tetraselmis extract to be an extract obtained from Tetraselmis suecica according to the previously described aspects.

Surprisingly, the combination of niacinamide and the Tetraselmis extract as described by the previous inventive aspects, especially when the contained Tetraselmis extract is prepared according to the second aspect of the invention, is especially effective when used as a medicament for treating or preventing dysfunctions of human hair and/or skin, acne vulgaris or seborrheic dermatitis.

Additionally, a Tetraselmis extract concentrate as described herein is especially preferred, which is used as a medicament for treating or preventing dysfunctions of human hair and/or skin, seborrhoeic dermatitis (seborrhea), acne vulgaris, wound healing, tissue regeneration, post-inflammatory hyperpigmentation, inflammatory related diseases, dandruff or Pityriasis versicolor. Treatment of Pityriasis versicolor is preferably achieved by reducing Malassezia.

Hereby, it is highly preferred for the Tetraselmis extract concentrate to be used as a medicament for treating or preventing dysfunctions of the human hair and/or skin, inflammatory related diseases or acnes, wherein it is most preferred for the Tetraselmis extract to be an extract obtained from Tetraselmis suecica according to the previously described aspects.

The Tetraselmis extract concentrate comprising the Tetraselmis extract or the combination composition as described in previous inventive aspects of the invention is found to be effective when used as a medicament for treating or preventing dysfunctions of the human hair and/or skin, inflammatory related diseases or acnes. Hereby, it is preferred for the Tetraselmis extract concentrate to contain a Tetraselmis extract obtained by the method claimed by the second aspect of the invention, as such a Tetraselmis extract concentrate is especially effective when used as a medicament for treating or preventing dysfunctions of the human hair and/or skin, inflammatory related diseases or acnes.

Further preferred is that the dermatological or therapeutic product according to the invention comprises a Tetraselmis extract or a combination composition or a Tetraselmis extract concentrate according to the invention, and optionally auxiliary substances, for use in treating skin diseases.

The preparations can also contain a solvent, such as the original extractant or preferably water in a quantity of up to 99 wt. %, preferably 5 to 80 wt. %, based on the total weight of the preparation. Hereby it is even more preferred for the formulations according to the invention to be a e.g. W/O (water-in-oil) emulsion, O/W (oil-in-water) emulsion, W/O/W (water-in-oil-in-water) emulsion, O/W/O (oil-in-water-in-oil) emulsion. The solvent may also be a solvent system in the amounts indicated above and may contain in parts glycerin.

Auxiliary substances and additives can be included in quantities of 0.1 to 99 wt. %, preferably 1 to 90 wt. %, preferably 60 to 80 wt. %, based on the total weight of the formulation.

It is preferred for the auxiliary substances and/or additives to be chosen from one or more of the groups of cooling agents, film-forming substances, antioxidants, vitamins, 2-hydroxycarboxylic acids, skin colouring agents, skin-moisturising substances, fats/fatty acids, waxes or other conventional constituents of a cosmetic or dermatological formulation such as alcohols, polyols, polymers, foam stabilisers, electrolytes, organic solvents, silicone derivatives or chelating agents, perfumes, substances to prevent foaming, dyes, pigments having a colouring action, thickeners, surface-active substances, emulsifiers, plant parts and plant extracts, animal extracts, propolis, proteins, protein hydrolysates and yeast extracts.

Hereby it is especially preferred for the film-forming substance to be chosen from e.g. polyvinyl pyrrolidones or chitosan or derivatives thereof;

for the vitamins to be chosen form e.g. vitamin C and derivatives, tocopherols and derivatives, vitamin A and derivatives;
for the 2-hydroxycarboxylic acids to be chosen form e.g. citric acid, malic acid, L-, D- or di-lactic acid;
for the skin colouring agents to be chosen from e.g. walnut extracts or dihydroxyacetone;
for the skin-moisturizing agents to be chosen form e.g. glycerol or urea;
for the fatty acids to be chosen from either of or combinations of the subgroups of monounsaturated or polyunsaturated fatty acids or α-hydroxy acids or polyhydroxy fatty acids or derivatives thereof such as e.g. linoleic acid, α-linolenic acid, γ-linolenic acid or arachidonic acid and the natural or synthetic esters thereof;
for the chelating agents to be chosen form e.g. ethylene diamine tetraacetic acid and derivatives;
for the thickeners to be chosen form silicon dioxide, aluminium silicates, such as e.g. bentonites, polysaccharides or derivatives thereof, e.g. hyaluric acid, guar gum, xanthan gum, hydroxypropyl methylcellulose or allulose derivatives, particularly advantageously polyacrylates such as e.g. carbopols or polyurethanes;
for the plant parts and plant extracts to be chosen from either or combinations of either of the plants e.g. arnica, aloe, beard lichen, ivy, stinging nettle, ginseng, henna, camomile, marigold, rosemary, sage, horsetail, oat, ginger, hop, wheat or thyme; when said compound group is employed as an auxiliary substance and/or additive.

In a further seventh invention aspect, we present a cosmetic product. Particularly preferred, is a cosmetic product comprising a Tetraselmis extract or a combination composition or a Tetraselmis extract concentrate according to the invention, and optionally auxiliary substances and/or perfumes, wherein the cosmetic product is a human skin and/or hair care product.

Further preferred is that the dermatological or therapeutic product as previous mentioned or cosmetic product according to the invention, comprises an amount of Tetraselmis extract or Tetraselmis extract concentrate in the product of 0.0001 to 10 wt. %, preferably 0.005 to 3 wt. % based on the total product weight. The weight ratios are calculated based on Tetraselmis extract dry weight.

Additionally, it is preferred for the Tetraselmis extract or the Tetraselmis extract concentrate employed in the dermatological or therapeutic product as described above to be prepared according to the second aspect of the invention. Furthermore, it is preferred for said Tetraselmis extract or said Tetraselmis extract concentrate to be prepared from Tetraselmis suecica.

In another preferred variation, the invention refers to a non-therapeutic or cosmetic use of a Tetraselmis extract or a combination composition or a Tetraselmis extract concentrate according to the invention for application, caring, cleansing, sun-protecting or protecting the skin and/or the hair.

Preferred compositions according to the present inventions are selected from the group of products for treatment, protecting, care and cleansing of the skin and/or hair or as a make-up product, as a leave-on or rinse-off product, most preferably as leave-on product.

The formulations according to the invention are preferably in the form of an emulsion.

Hereby it is even more preferred for the formulations according to the invention to be a e.g. W/O (water-in-oil) emulsion, O/W (oil-in-water) emulsion, W/O/W (water-in-oil-in-water) emulsion, O/W/O (oil-in-water-in-oil) emulsion, PIT emulsion, Pickering emulsion, emulsion with a low oil content, micro- or nanoemulsion, a solution, e.g. in oil (fatty oils or fatty acid esters, in particular C₆-C₃₂-fatty acid, C₂-C₃₀-esters or silicone oil, dispersion, suspension, creme, lotion or milk, depending on the production method and ingredients, a gel (including hydrogel, hydrodispersion gel, oleogel), spray (e.g. pump spray or spray with propellant) or a foam or an impregnating solution for cosmetic wipes, a detergent, e.g. soap, synthetic detergent, liquid washing, shower and bath preparation, bath product (capsule, oil, tablet, salt, bath salt, soap, etc.), effervescent preparation, a skin care product such as e.g. an emulsion (as described above), ointment, paste, gel (as described above), oil, balsam, serum, powder (e.g. face powder, body powder), a tonic, a mask, a pencil, stick, roll-on, pump, aerosol (foaming, non-foaming or post-foaming), a deodorant and/or antiperspirant, mouthwash and mouth rinse, a foot care product (including keratolytic, deodorant), an insect repellent, a sunscreen, after sun preparation, a shaving product, aftershave balm, pre- and aftershave lotion, a depilatory agent, a hair care product such as e.g. shampoo (including 2-in-1 shampoo, anti-dandruff shampoo, baby shampoo, shampoo for scalps, concentrated shampoo), conditioner, hair tonic, hair water, hair rinse, styling creme, pomade, perm and setting lotion, hair spray, styling aid (e.g. gel or wax), hair smoothing agent (detangling agent, relaxer), hair dye such as e.g. temporary direct-dyeing hair dye, semi-permanent hair dye, permanent hair dye, hair conditioner, hair mousse, eye care product, make-up, make-up remover or baby product.

The formulations according to the invention are particularly preferably in the form of an emulsion, in particular in the form of a W/O, O/W, W/O/W, O/W/O emulsion, PIT emulsion, Pickering emulsion, emulsion with a low oil content, micro- or nanoemulsion, a gel (including hydrogel, hydrodispersion gel, oleogel), a detergent (e.g. soap, synthetic detergent, liquid washing), a solution (e.g. tonic, facial toner or as impregnating solution for wet wipes), a spray (e.g. pump spray or spray with propellant) or a shampoo (including 2-in-1 shampoo, anti-dandruff shampoo, baby shampoo, shampoo for sensitive scalps, concentrated shampoo), conditioner, hair tonic, hair mask or hair water.

In another preferred eighth aspect, we describe the cosmetic use of a Tetraselmis extract or a combination composition or a Tetraselmis extract concentrate according to the invention for reduction of sebum.

A further preferred ninth aspect of the invention is the use of a Tetraselmis extract or a combination or a Tetraselmis extract concentrate according to the invention for:

a) stimulation of cutaneous junctions,
b) stimulation of cutaneous antimicrobial peptides,
c) reduction of COX-2 gene expression and prostaglandin mediated effects,
d) reduction of post-inflammatory hyperpigmentation,
e) stimulation of filaggrin.

Preferably, the Tetraselmis extract or combination composition or Tetraselmis extract concentrate according to the invention is used cosmetically:

a) for improvement of epidermal integrity of the skin,
b) for prevention of external stimuli such as air pollution or particulate matter induced effects,
c) for prevention of skin barrier dysfunction.

An alternative preferred variation is the therapeutic or cosmetic product according to the invention, further comprising one or more of the following: other sebum reducing agents, anti-acne agents, anti-dandruff agents, other anti-inflammatory agents, TRPV1 antagonists, anti-itch agents, anti-microbial agents, especially anti-Propionibacterium acnes agents, anti-Malassezia agents.

We presently also disclose a therapeutic product according to the previous variation for use as a medicament in the treatment of any disease as described herein according to the invention, in particular for skin diseases.

We presently also disclose a cosmetic product as mentioned above for use in a non-therapeutic application as described herein according to the invention, in particular for skin protection.

In formulations, the Tetraselmis extract may be combined with other sebum reducers and/or anti-acne agents especially if these act via different pathways as thus a more pronounced activity can be expected. Since the seborrhoeic condition of the skin is an ideal nutrient medium for bacterial and fungal growth and consequently for e.g. the development of impure skin or acne, a composition for prophylaxis and/or treatment of oily skin is likewise a preferred composition for prophylaxis and/or treatment of impure skin or acne. Suitable agents are e.g. retinoids like 13-cis retinoic acid (isotretinoin), all-trans-retinoic acid, adapalene, their salts or derivatives, androgen inhibitors like spironolactone and cyproterone, antibiotics, preferably clindamycin, erythromycin and tetracycline, zinc or zinc salts, and antiandrogens, 5-alpha-reductase inhibitors, D-panthenol, alpha-hydroxy acids, such as e.g. salicylic acid and lactic acid, pyruvic (alfa-keto acid) acids, aliphatic dicarboxylic acids, such as e.g. azelaic acid, L-carnitine, bakuchiol, 1,2-decanediol, senkyunolide-A and senkyunolide-A containing Apium graveolens seed oil, Quillaja saponaria extract, Enantia chlorantha bark extract, Spiraea ulmaria extract, butyl avocate, vitamin B6 (also known as pyridoxine) or its salts or derivatives, vitamin B3 (also known as niacin or nicotinic acid) or its salts or derivatives, benzoylperoxide, phloretin, Camellia sinensis extract and contained polyphenols such as e.g. epigallocatechin-3-gallate, red clover (Trifolium pretense) extract, soybean (Glycine soja) seed extract, isoflavonoids or isoflavonoid containing extracts, preferably biochanin A, genistein, daidzein, genistin, and daizin.

The abovementioned product groups, preferably in combination with the preferred auxiliary substances, additives and/or active compounds for formulations for the reduction of the sebum concentration of the skin are also preferred as formulations for prophylaxis and/or treatment of oily skin, impure skin or acne.

A preferred cosmetic or therapeutic dermatological formulation for topical application comprises the following constituents or consists of the following: an amount of Tetraselmis, in particular Tetraselmis suecica which is sufficient to reduce the sebum concentration of the skin as well as one or more active compounds. More preferably said formulation comprises a combination of two, three or four active compounds.

Preferably, the active compounds are chosen from one or more of the compound classes in the following group: antiandrogens, isoflavonoid containing extracts, retinoids, vitamins, organic peroxides, organic ethers, organic acids or alcohols.

More preferably, the active components are chosen from: 1,2-decanediol, bakuchiol, salicylic acid; lactic acid; azelaic acid; retinoids, preferably 13-cis retinoic acid (isotretinoin), all-trans-retinoic acid, adapalene, their salts or derivatives; benzoyl peroxide; D-panthenol, vitamin B6 (also known as pyridoxine) or its salts e.g. pyridoxine.HCl or derivatives, vitamin B3 (also known as niacin or nicotinic acid) or its salts or derivatives, butyl avocadate, farnesol; phenoxyethanol; red clover (Trifolium pretense) extract, isoflavonoids or isoflavonoid containing extracts, preferably biochanin A, genistein, daidzein, genistin and daizin, and antiandrogens, preferably 5-alpha-reductase inhibitors.

Even more preferably, the one or more active compounds are chosen from the group consisting of: 1,2-decanediol, salicylic acid, lactic acid, azelaic acid, benzoyl peroxide, D-panthenol, 13-cis retinoic acid (isotretinoin), all-trans-retinoic acid, adapalene, their salts or derivatives, bakuchiol, erythromycin, sulfur, butyl avocadate, farnesol, phenoxyethanol, pyridoxine.HCl, red clover (Trifolium pretense) extract, biochanin A, genistein, daidzein, genistin, daizin and 5alpha-reductase inhibitor.

Even more preferred, the one or more active compounds are chosen from the group consisting of: 1,2-decanediol, salicylic acid, azelaic acid, benzoyl peroxide, D-panthenol, 13-cis retinoic acid (isotretinoin), all-trans-retinoic acid, adapalene, their salts or derivatives, bakuchiol, erythromycin, butyl avocadate, phenoxyethanol, pyridoxine.HCl, red clover (Trifolium pretense) extract, biochanin A, genistein, daidzein, and 5-alpha-reductase inhibitor.

Most preferred, the one or more active compounds are chosen from the group consisting of: 1,2-decanediol, salicylic acid, azelaic acid, benzoyl peroxide, D-panthenol, adapalene, bakuchiol, erythromycin, butyl avocadate, pyridoxine.HCl, and biochanin A.

Furthermore, it is highly preferred to include niacinamide as an active compound.

Preferably the one or more active compounds are combined with anti-dandruff active agents. A more pronounced overall effect is found especially if these act via different biological pathways. Anti-dandruff agents may be one material or a mixture selected from the groups consisting of: azoles, such as climbazole, ketoconazole, itraconazole, econazole, and elubiol; hydroxy pyridones, such as octopirox (piroctone olamine), ciclopirox, rilopirox, and MEA-hydroxyoctyloxypyridinone; kerolytic agents, such as salicylic acid and other hydroxy acids; strobilurins such as azoxystrobin and metal chelators such as 1,10-phenanthroline.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof.

In an embodiment, the preferred anti-dandruff agents may be present in an amount from 0.1 wt. % to 10 wt. %, in a further embodiment from 0.25 wt. % to 8 wt. %, in yet a further embodiment from 0.5 wt. % to 6 wt. %.

The products and concentrates derived or based on Tetraselmis extracts according to the invention may be combined with sun protection factors, for example, organic substances (light filters) which are liquid or crystalline at room temperature and which are capable of absorbing ultraviolet radiation and of releasing the energy absorbed in the form of longer-wave radiation, for example heat or inorganic UV filters such as titanium dioxide (TiO₂) or zinc oxide (ZnO).

Preferred cosmetic compositions and products, preferably topical formulations according to the present invention comprise one, two, three or more sun protection factors selected from the group consisting of 4-aminobenzoic acid and derivatives, salicylic acid derivatives, benzophenone derivatives, dibenzoylmethane derivatives, diphenyl acrylates, 3-imidazol-4-yl acrylic acid and esters thereof, benzofuran derivatives, benzylidene malonate derivatives, polymeric UV absorbers containing one or more organosilicon radicals, cinnamic acid derivatives, camphor derivatives, trianilino-s-triazine derivatives, 2-hydroxyphenylbenzotriazole derivatives, phenylbenzimidazole sulfonic acid derivatives and salts thereof, anthranilic acid menthyl esters, benzotriazole derivatives and indole derivatives.

The formulations and products according to the invention advantageously contain at least one UV-A filter and/or at least one UV-B filter and/or a broadband filter and/or at least one inorganic pigment. Formulations according to the invention preferably contain at least one UV-B filter or a broadband filter, more particularly preferably at least one UV-A filter and at least one UV-B filter.

In formulations and products according to the invention, the Tetraselmis extract may also be combined with anti-inflammatory or anti-irritant agents, preferably if these agent act via different pathways than COX-2/PGE2 and/or anti-acne agents and/or anti-microbial agents effecting acne-related P. acnes and/or dandruff related Malassezia sp. These combinations are especially beneficial if the formulation is intended for use on impure, acne-prone or acne oily skin or sensitive oily skin or sensitive oily scalp or dandruff.

The compositions and products of the invention may contain anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives, in particular avenanthramides described in WO 2004 047833 A1, are preferred anti-itch ingredients in a composition according to the present invention.

Also useful are natural or naturally occurring anti-inflammatory/anti-irritant mixtures of substances or mixtures of substances that alleviate inflammation and/or reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, natural alpha-bisabolol, synthetic bisabolol, apigenin, apigenin-7-glucoside, gingerols, shogaols, gingerdiols, dehydrogingerdiones, paradols, especially natural or synthetic 6-paradol, naturally occurring avenanthramides, preferably avenanthramide A, avenanthramide B, avenanthramide C, avenanthramide D, avenanthramide E, non-natural or non-naturally occurring avenanthramides, preferably dihydroavenanthramide D, dihydroavenanthramide E, tranilast, boswellic acid, phytosterols, glycyrrhizin, glabridin, sclareolide and licochalcone A; preferably selected from the group consisting of natural alpha-bisabolol, synthetic bisabolol, natural avenanthramides, non-natural avenanthramides, preferably dihydroavenanthramide D (as described in WO 2004 047833 A1), ginger extract, gingerols, shogaols, gingerdiols, dehydrogingerdiones, paradols, especially natural or synthetic 6-paradol, boswellic acid, phytosterols, glycyrrhizin, and licochalcone A, and/or allantoin, sclareolide, panthenol, (pseudo-)ceramides [preferably Ceramide 2, hydroxypropyl bispalmitamide M EA, cetyloxypropyl glyceryl methoxypropyl myristamide, N—(I-hexadecanoyl)-4-hydroxy-L-proline (1-hexadecyl) ester, hydroxyethyl palmityl oxyhydroxypropyl palmitamide], phytosterols, chitosan, and β-glucans, in particular 1,3-1,4-glucan from oats.

The total amount of anti-irritants or anti-inflammatory substances in a formulation or product according to the invention is preferably in the range of from 0.0001 to 20 wt %, preferably from 0.0001 to 10 wt %, in particular from 0.001 to 5 wt %, based on the total weight of the formulation or product, respectively.

Transient receptor potential cation channel subfamily V member 1 (TRPV1) antagonists

Suitable compounds that can be combined with the products of the invention are such which reduce the hypersensitivity of skin nerves based on their action as TRPV1 antagonists, these encompass preferably e.g. trans-4-tert-butyl cyclohexanol as described in WO 2009 087242 A1, or indirect modulators of TRPV1 by an activation of the μ-receptor, e.g. acetyl tetrapeptide-15.

Tetraselmis extracts in the inventive formulations may also be combined anti-dandruff agents. Suitable anti-dandruff agents are Pirocton Olamin (1-hydroxy-4-methyl-6-(2,4,4-trimethylpentyl)-2-(1H)-pyridinone monoethanolamine salt), Baypival (Climbazole), Ketoconazol® (2RS,4SR)-1-(4-{4-[-2-(2,4-Dichlorphenyl)-2-(imidazol-1-ylmethyl)-1,3-dioxolan-4-ylmethoxy]phenyl}piperazin-1-yl)ethanon, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon S (protein/undecylenic acid condensate), zinc pyrithione, aluminium pyrithione and magnesium pyrithione/dipyrithione magnesium sulfate.

A further preferred cosmetic formulation for topical application comprises the following constituents or consists of the following constituents:

an amount of Tetraselmis extract which is sufficient to reduce the sebum concentration of the skin;

one, two, three, four, five, six, seven, eight, nine, ten or more, preferably two or more, more preferably three or more cleansing auxiliary substances;

optionally one or more further auxiliary substances and/or additives.

Such a cosmetic formulation is particularly suitable for cleansing greasy-oily and/or impure skin.

In formulations, the inventive Tetraselmis extracts and products may also be combined with film formers especially as these provide an additional topical, physical barrier to protect the skin. They will add to the epidermal-integrity-improving effect of Tetraselmis extract, which is especially beneficial as external stimuli such as e.g. PM were shown to increase sebum production and lead to barrier dysfunction.

Typical film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof, beta-glucans like 1,3-1,4-glucan from oats or 1,3-1,6-glucans from yeasts or mushrooms and similar compounds.

### EXPERIMENTAL SECTION

### Example 1: Preparation of a Tetraselmis suecica Extract

3 g freeze-dried Tetraselmis suecica biomass and 30 g of water were mixed and stirred for 2 hours at 80° C. The liquid extract was separated from the biomass, 30 g of water was added to the extracted biomass and the mixture was stirred for another 2 hours at 80° C. The liquid was separated from the biomass by centrifugation, both extract solutions were combined, and the water was removed by freeze-drying. The extractions were performed with 3 different biomass batches.

For comparison, an aqueous extract according to the description in US2010143267 A1 was prepared from the same 3 biomass batches and water was removed by freeze-drying.

TABLE 1
Tetraselmis suecica extract obtained by extraction
at room temperature and at 80° C.

| Condition of extraction | Mean yield | Appearance |
|---|---|---|
| 80° C. | 38.4 ± 0.2% | Beige greenish solid |
| Room temperature | 40.0 ± 0.9% | Intensive dark green solid |
| (18 to 23° C.) | | |

Extraction upon heating gives a well comparable, very slightly lower, high extraction yield when compared to extraction at room temperature, but it surprisingly gives a much lighter colored extract which is especially advantageous for the use as cosmetic ingredient as consumers prefer low colored products. Heat treatment furthermore has the additional advantage that enzymes in the biomass are inactivated which is especially advantageous when using viable or non-inactivated biomass. Additionally, microbiological contamination by bacteria, fungi or yeasts, which is especially challenging for extractions with water or extractant systems with high water content at low temperatures is prevented by extracting at higher temperatures (>50° C.)

TABLE 2
Composition of Tetraselmis suecica extract
obtained by extraction at 80° C.

| | Mean content | Mean content |
|---|---|---|
| | [wt.-%] | [wt.-%] |
| | (Extract | (Extract obtained at |
| | obtained | Room temperature |
| Substance class | at 80° C.) | (18 to 23° C.)) |
| Sum of minerals | 20.3 ± 0.6 | 21.4 ± 0.8 |
| Containing but not limited to: | | |
| Sodium Na⁺ | 5.7 ± 0.2 | 5.7 ± 0.3 |
| Potassium K⁺ | 3.6 ± 0.1 | 3.6 ± 0.2 |
| Magnesium Mg²⁺ | 0.7 ± 0.1 | 0.7 ± 0.0 |
| Calcium Ca²⁺ | 0.8 ± 0.1 | 0.9 ± 0.0 |
| Chloride Cl⁻ | 6.9 ± 0.2 | 7.1 ± 0.2 |
| Sulfate SO₄²⁻ | 2.1 ± 0.1 | 2.6 ± 0.1 |
| Phosphate PO₄³⁻ | 0.4 ± 0.2 | 0.9 ± 0.4 |
| Mannitol | 11.8 ± 0.9 | 11.6 ± 0.7 |
| Total galactose (free and bound)* | 9.7 ± 0.7 | 7.8 ± 0.4 |
| Total glucose (free and bound)* | 7.0 ± 0.8 | 3.5 ± 0.0 |
| Sum of amino acids | 8.4 ± 0.8 | 9.2 ± 0.7 |
| Containing but not limited to: | | |
| Glutamic acid | 2.88 ± 0.30 | 2.77 ± 0.27 |
| Alanine | 1.11 ± 0.08 | 1.10 ± 0.10 |
| Arginine | 0.80 ± 0.30 | 0.17 ± 0.02 |
| Ornithine | 0.54 ± 0.28 | 1.26 ± 0.17 |
| Citruline | 0.53 ± 0.23 | 0.74 ± 0.03 |
| Asparagine | 0.39 ± 0.04 | 0.19 ± 0.02 |
| Taurine | 0.39 ± 0.04 | 0.35 ± 0.04 |
| Lysine | 0.37 ± 0.04 | 0.49 ± 0.02 |
| Aspartic acid | 0.27 ± 0.10 | 0.76 ± 0.03 |
| Proline | 0.14 ± 0.02 | 0.18 ± 0.02 |
| Glutamine | 0.12 ± 0.06 | 0.07 ± 0.02 |
| Total Nitrogen** | 4.22 ± 0.17 | 4.91 ± 0.16 |

*determined after hydrolysis and derivatization by GC
**determined by nitrogen analyzer

In comparing the values for the extract at RT and at 80° C., it is apparent that certain components are conserved whereas others are shifted respective the lower temperature extraction. In particular, the content of galactose and glucose differs significantly (is increased from 7.8 to 9.7 wt.-% and 3.5 to 7.0 wt-%, respectively). Also, the content of certain amino acids is significantly enhanced, such as Arginine and Asparagine (0.8 from 0.17 wt.-% and 0.39 from 0.19 wt.-%, respectively). Some other amino acids are selectively decreased such as Aspartic acid which drops from 0.76 wt.-% to 0.27 wt.-% and Ornithine which drops from 1.26 wt.-% to 0.54 wt.-%. Overall most mineral compounds are conserved except for phosphate.

### Example 2: Preparation of Liquid Versions of Tetraselmis suecica Extract

To 4.6 g Tetraselmis suecica extract dry matter obtained by extraction at 80° C. according to Example 1, 97 g water, 79 g glycerin (99.5%), 0.5% sodium benzoate and 0.2% potassium sorbate (both based on the total weight of the liquid mixture) were added, and the pH of the mixture was adjusted with help of lactic acid to 4.5 giving a yellow beige to light brownish solution, refractive index (n₂₀/D): 1.396, mannitol content: 0.29%.

For another liquid version, 25 g Tetraselmis suecica extract dry matter obtained by extraction at 80-90° C. according to Example 1, was dissolved in 483 g water and 392 g glycerin (99.5%) and 100 g of 1,2-pentanediol (Hydrolite-5) were added. A light yellow-greenish, clear to slightly turbid solution was obtained; color according to L*a*b* color system: L* 88.4, a* −13.6, b* 47.5, pH 7.6, mannitol content: 0.28%.

### Example 3: Effect of Tetraselmis suecica Extract (Dried) on the Total Lipid Content of Ex Vivo Human Sebaceous Glands

Organ culture of human sebaceous glands micro-dissected from human skin explants was performed to evaluate the modulatory activity of Tetraselmis suecica extract prepared according to the description given in Example 1 on the sebum level. The extract is employed in the dried form.

After removal of the epidermis of the full thickness skin sample, the sebaceous glands were carefully removed using micro-scissors and scalpel. The micro-dissected sebaceous glands were then pooled in groups of 8 and cultured up to day 6 in a 24 well plate immersed in 500 μl of modified Williams' E medium. After 24 hours of acclimation the culture medium was changed and substituted with the medium containing the extract to be tested. The medium was renewed at day 3 and 5 of culture. At day 6 the glands were collected and used for the quantification of lipids and proteins. In order to make the estimated productivity of the glands comparable, which are variable in biomass, their total sebum content was estimated and divided by the proteins extracted from the gland tissue, obtaining the ratio between the produced sebum and the tissue proteins (i.e. mg of lipids/mg of proteins).

To do so, each sebaceous glands group was homogenized in 100 μl of isopropyl alcohol to extract lipids and let the proteins undissolved. After centrifugation the supernatant containing the extracted sebum was collected and analyzed. The remaining pellet was dried using a vacuum dry evaporator and then minced in presence of 50 μl of protein lysis buffer. After an appropriate incubation time, this extractive mixture was centrifuged, and the supernatant was collected and analyzed. The lipids dissolved in isopropyl alcohol and the proteins dissolved in the lysis buffer were quantified by infrared spectroscopy using a Direct Detect IR Spectrometer (Millipore). The total lipid amount was obtained by normalizing the quantified lipids upon the quantified proteins (i.e. mg of lipids/mg of proteins). The amounts of normalized lipids, i.e. the sebum produced by each group of sebaceous glands, obtained from the treated groups was compared to that of the untreated control group and the modulatory activity was calculated in percentage. As positive control, a 5 μM Capsaicin treatment was included in the experimental design. Capsaicin is an active component of chili peppers suitable to inhibit sebogenesis [Tóth et al., J. Invest. Derm. (2009), 129: 329-339]. For statistical analysis, differences among groups were evaluated by one-way anova with permutation test followed by Dunnett's permutation test.

To better understand the response to the extract, a viability test was performed in parallel at day 1 and day 6 of organ culture. Resazurin was added to the wells (1:11) and let incubate for 2 hours. At the end of the incubation an aliquot of the medium was read with a fluorometer (excitation: 560 nm, emission: 590 nm). The medium was then replaced with normal medium for 2 hours in order to eliminate residual resazurin. After this the medium was replaced again with medium containing the test samples. The viability in each well was measured as the difference in percentage between day 6 and day 1.

To evaluate donor responsiveness and interindividual variability the extract was tested on sebaceous glands obtained from skin samples of three different donors.

TABLE 3
Effect of Tetraselmis suecica water-extract (dried)on lipids and
viability of micro-dissected human sebaceous glands

| Parameter | Test sample | Donor 1 | Donor 2 | Donor 3 |
|---|---|---|---|---|
| Reduction of lipids | 5 μM (=1.5 ppm) | 11 | 28 | 14 |
| at day 6 versus | Capsaicin | | | |
| untreated [%]* | 0.3 ppm extract | 19 | 33 | 18 |
| | (extraction at 80° C.) | | | |
| Viability [%] | Untreated | 93 | 100 | 81 |
| | 5 μM (1.5 ppm) | 92 | 99 | 85 |
| | Capsaicin | | | |
| | 0.3 ppm extract | 108 | 101 | 83 |
| | (extraction at 80° C.) | | | |

*All results were statistically significant versus untreated with p < 0.01

In the present cell tests, ex vivo and in vitro, and generally for biological tests, the dried form of the Tetraselmis extract is employed to avoid side effects resulting from solvents, glycerin or the preservative system.

The results show that Tetraselmis suecica water extract (dried) obtained by extraction at 80° C. is surprisingly a highly effective reducer of the normalized total lipids, i.e. sebum content of human sebaceous glands without affecting their viability. It is more effective than the positive control capsaicin and this even at a 5-fold lower concentration. Furthermore, the sebaceous glands obtained from all three donors responded to the extract (donor responsiveness: 100%).

### Example 4: Synergistic Effect of Tetraselmis suecica Extract (Dried) and Niacinamide on the Total Lipid Content of Ex Vivo Human Sebaceous Glands

The same experimental set-up as described in Example 3 was used to evaluate the combination of Tetraselmis suecica extract and niacinamide for synergistic activity.

Kull's equation for calculation of the synergism index SI was used:

SI=C×D/A+C×E/B

### With

A=lipid reduction by Tetraselmis suecica extract at concentration x
B=lipid reduction by niacinamide at concentration y
C=lipid reduction by the combination of Tetraselmis suecica extract at concentration x/2 and niacinamide at concentration y/2
D=Factor for Tetraselmis suecica extract=>0.5 (due to half concentration tested in the combination)
E=Factor for niacinamide=>0.5 (due to half concentration tested in the combination)
A SI=1 is obtained for additive activity of the two combined components, whereas a SI<1 proves antagonistic activity (observed efficacy is lower than additive) and SI>1 proves synergistic activity (observed efficacy is higher than additive). Results of this experiment are summarized in Table 4.

TABLE 4
Tetraselmis suecica extract and niacinamide on the total lipid
content of ex vivo human sebaceous glands

| Parameter | Test sample | Donor 1 |
|---|---|---|
| Reduction of lipids at day 6 | 0.3 ppm extract | 11 |
| versus untreated [%]* | 100 ppm niacinamide | 6 |
| | 0.15 ppm extract + | 13 |
| | 50 ppm niacinamide | |
| | Untreated | 81 |
| Viability [%] | 0.3 ppm extract | 79 |
| | 100 ppm niacinamide | 78 |
| | 0.15 ppm extract + | 81 |
| | 50 ppm niacinamide | |

*All results were statistically significant versus untreated with p < 0.01 SI = 13 × 0.5/11 + 13 × 0.5/6 = 1.674

The obtained SI of 1.674 clearly proves that a combination of Tetraselmis suecica water extract and niacinamide surprisingly synergistically reduce the total lipids content, i.e. sebum level of human sebaceous glands.

### Example 5: Effect of Tetraselmis suecica Extract (Dried) on the Gene Expression of Human Sebocytes

Dermal primary human sebocytes (from face (T-zone) localization, Caucasian donor, purchased from Zen-bio) were cultivated in sebum basal medium at 5% CO₂ at 37° C. according to the supplier instructions. Sebocytes were treated for 24 hours with Tetraselmis suecica water extract obtained according to Example 1 by extraction at 80° C. at 0.01% and 0.1% or DMSO as vehicle control. Each experiment was performed in triplicate. Genomic target expression levels in extract treated cells were measured by RT-qPCR comparing to DMSO treatment.

Total RNA with miRNAs from sebocytes stimulated with the extract over 24 h, was extracted and purified using Qiaquick RNA Isolation Kit (from Quiagen), following the manufacturer's instructions. For mRNA target quantitation, total RNA was reverse-transcribed with the Superscript VILO cDNA Synthesis Kit (ThermoFisher) according to the manufacturer's instructions. The purity of the isolated RNA was determined by spectrophotometry: ratio 260/280≥1.5 to 2 (RNA extract is free of protein contamination). RQ values were calculated and the results were normalized to endogenous control GAPDH expression. Statistical analysis was performed using two-tailed unpaired T-test (*p-value<0.05). Results of this experiment are summarized in Table 5.

TABLE 5
Modulation of gene expression of primary human sebocytes after
treatment with Tetraselmis suecica water dry extract

| | Not treated | 0.01% | |
|---|---|---|---|
| Genes/RQ values per | (DMSO | extract | 0.1% extract |
| treatment | control) | (p value) | (p value) |
| SREBF1 (SREBP-1) [sterol | 1.00 ± 0.04 | 0.81 ± 0.03 | 0.59 ± 0.03 |
| regulatory element binding | | (p < 0.05) | (p < 0.001) |
| transcription factor 1] | | | |
| DGAT1 [diacylglycerol O- | 1.01 ± 0.08 | 0.81 ± 0.04 | 0.69 ± 0.03 |
| acyl-transferase 1] | | (n.s.) | (p < 0.05) |
| MGAT1 [mannosyl (alpha- | 1.00 ± 0.05 | 0.76 ± 0.01 | 0.52 ± 0.04 |
| 1,3-)-glycoprotein beta-1,2-N- | | (p < 0.05) | (p < 0.01) |
| acetylglucosaminyltransferase] | | | |
| SCD [stearoyl-CoA | 1.01 ± 0.09 | 0.92 ± 0.06 | 0.59 ± 0.02 |
| desaturase] | | (n.s.) | (p < 0.05) |
| PTGS2 (COX-2) | 1.01 ± 0.09 | 0.44 ± 0.02 | 0.20 ± 0.01 |
| [prostaglandin-endoperoxide | | (p < 0.01) | (p < 0.001) |
| synthase 2] | | | |
| NR1H3 (LXRa) [nuclear | 1.01 ± 0.10 | 0.81 ± 0.07 | 0.64 ± 0.03 |
| receptor subfamily 1 group H] | | (n.s.) | (p < 0.05) |
| APOC1 | 1.01 ± 0.07 | 0.73 ± 0.05 | 0.76 ± 0.07 |
| [apolipoprotein C1] | | (p < 0.05) | (p = 0.067) |
| ACAT1 | 1.01 ± 0.09 | 0.66 ± 0.05 | 0.73 ± 0.06 |
| [acetyl-CoA acetyltransferase | | (p < 0.05) | (p = 0.064) |
| 1] | | | |
| APPL1 [adaptor protein, | 1.00 ± 0.04 | 0.63 ± 0.05 | 0.73 ± 0.07 |
| phosphotyrosine interacting | | (p < 0.01) | (p < 0.05) |
| with PH domain and leucine | | | |
| zipper 1] | | | |
| ADIPOR1 | 1.00 ± 0.07 | 0.72 ± 0.02 | 0.72 ± 0.06 |
| [adiponectin receptor 1] | | (p < 0.05) | (p < 0.05) |

The results clearly show that Tetraselmis suecica water extract repress a large majority of genes involved in fatty acid, triglycerides and cholesterol production and thereby reduce the lipid, i.e. sebum production of sebocytes. The major pathways analyzed and known to regulate lipid synthesis and storage and sebaceous gland size, are involved.

Tetraselmis suecica water dry extract is able to modulate genes involved in lipid production and storage such as: fatty acid (SREBF1, SCD, APOC1), triglycerides (DGAT1) and cholesterol (ACAT1) and to regulate dedicated pathways including: adiponectin (ADIPOR1, APPL1), LXR/RXR/PPARA (SREBPF1, NH1H3 (LXRa), ACAT1, and prostaglandin (PTSG2 (COX2)).

Treatment with Tetraselmis suecica water extract at 0.01% statistically downregulated 7 genes (APOC1, SREBPF1, APPL1, ADIPOR1, MGAT1, ACAT1, PTSG2 (COX-2)).

Treatment with Tetraselmis suecica water dry extract at 0.1% statistically downregulated 8 genes (SREBF1, NR1H3 (LXRa), APPL1, ADIPOR1, DGAT1, MGAT1, SCD, PTSG2 (COX-2)).

The extract is able to reduce lipid production by repressing genes involved in fatty acid production (SREBF1, SCD, APOC1), diglycerides (MGAT1), triglycerides (DGAT1) and cholesterol (ACAT1).

IGF-I plays a key role in the induction of lipid synthesis in human sebocytes. In SEB-1 sebocytes, IGF-I increases lipogenesis by the induction of SREBF1 which preferentially regulates genes of fatty acid synthesis.

SCD is highly expressed in the sebaceous gland; SCD is a Δ9 fatty acid desaturase that primarily catalyzes the conversion of the saturated fatty acids palmitic acid (16:0) and stearic acid (18:0) into the cis-monounsaturated fatty acids (MUFA) palmitoleic acid (16:1n7) and oleic acid (18:1n9), respectively. The MUFA serve as important esterification substrates in the formation of triglycerides, cholesterol esters and wax esters, which are components of sebum.

Transgenic mice that overexpressed the APOC1 had hypoplastic sebaceous glands and hypertriglyceridemia.

DGAT1 catalyzes the final and rate-limiting step in triglyceride synthesis.

MGAT1 is involved in the synthesis of protein-bound and lipid-bound oligosaccharides. Acyl-CoA:monoacylglycerol acyltransferase (MGAT) genes are best known for their role in fat absorption in the intestine. MGAT1 has been shown to exhibit MGAT activity in mammalian cell lines, specific for catalyzing diacylglycerol synthesis by incorporating fatty acyl-CoA into diacylglycerol.

ACAT1 is an enzyme that catalyzes the formation of cholesteryl ester from free cholesterol and is highly expressed in the sebaceous gland, where it allows for the incorporation of cholesteryl esters into cytoplasmic lipid droplets.

The major pathways that induce lipid production and storage are LXR/RXR/PPARA and adiponectin. Indeed, it was shown that treatment of SZ95 sebocytes with LXR ligands enhanced accumulation of lipid droplets in the cells and lipid synthesis was markedly enhanced in sebocytes treated with adiponectin. Thus, NR1H3 (LXRa) that codes for a nuclear receptor responsible for activation of LXR/RXR/PPARA pathway is repressed by the extract.

The adiponectin receptor ADIPOR1 and its ligand APPL1 are responsible for activation of adiponectin pathway, are also reduced. In 3D culture of sebocytes, lipid synthesis was markedly enhanced in sebocytes treated with adiponectin.

The pronounced effect was surprisingly observed on the gene expression level of PTGS2 (COX-2). PTGS2 (COX-2) plays a major role in sebocyte function. Transgenic mice that overexpressed cyclooxygenase-2 (COX-2) exhibited an increased sebum level due to sebaceous gland hyperplasia. Thus, reduction of PTGS2 (COX-2) can be expected to lead to reduction of sebaceous gland size and sebum production.

### Example 6: In Viva Sebum Reduction by Tetraselmis suecica Extract (Dried)

A randomized, split-face study with 2 groups of 15 Caucasian volunteers each was performed. Volunteers applied the test products at home on both semi-foreheads twice daily (mornings and evenings) for 4 weeks. Test product was a hydrodispersion gel with and without 0.05% Tetraselmis suecica extract prepared by extraction at 80° C. according to Example 1. As positive control/reference a combination of 2% niacinamide and 1% D-panthenol (Z. D. Draelos et al. J. Cosmet. Laser Ther. 2006, 8:2, 96-101) formulated in hydrodispersion gel was used. Read-outs were Casual sebum level (sebumeter), sebum surface percentage and number of active pores (both Visioscan® equipped with a Sebufix® foil) and readings were performed at baseline (t₀) and after 4 weeks (t₁).

TABLE 6
Mean modulation of casual sebum level, sebum surface
percentage and number of active pores

| Test product/ | | | | |
|---|---|---|---|---|
| Hydrodispersion gel | | | Modulation | |
| with | T0 | T1 | t₁ versus t₀ | Signif- |
| Casual sebum Level | (baseline) | (4 weeks) | in % | icance |
| No active (Placebo) | 176.1 ± 40.4 | 166.5 ± 42.5 | −5.5 | n.s. |
| 0.05% Tetraselmis | 186.4 ± 36.5 | 166.2 ± 40.4 | −10.8 | p < |
| suecica extract | | | | 0.05 |
| 2% Niacinamide and | 180.4 ± 40.0 | 152.4 ± 48.1 | −15.6 | p < |
| 1% D-panthenol | | | | 0.05 |
| Sebum surface | | | | |
| percentage | | | | |
| No active (Placebo) | 10.3 ± 6.3 | 10.3 ± 7.3 | −0.4 | n.s. |
| 0.05% Tetraselmis | 12.2 ± 8.6 | 8.4 ± 6.6 | −31.3 | p < |
| suecica extract | | | | 0.05 |
| 2% Niacinamide and | 10.1 ± 8.2 | 9.0 ± 5.8 | −11.7 | n.s. |
| 1% D-panthenol | | | | |
| Number of active pores | | | | |
| No active (Placebo) | 175.3 ± 47.0 | 149.5 ± 52.4 | −14.7 | n.s. |
| 0.05% Tetraselmis | 174.8 ± 61.9 | 129.8 ± 64.8 | −25.7 | p < |
| suecica extract | | | | 0.05 |
| 2% Niacinamide and | 154.4 ± 50.9 | 136.6 ± 65.7 | −11.6 | n.s. |
| 1% D-panthenol | | | | |

n.s. = non-significant

The results clearly proof the potent sebum reducing activity of Tetraselmis suecica water extract also in vivo. The extract is the only active which significantly reduced all three read-outs (casual sebum level by −10.8%, sebum surface percentage by −31.3% and number of active pores by −25.7%). The positive control/reference, the combination of 2% Niacinamide and 1% D-panthenol, significantly reduced the casual sebum level by −16.5%, but failed to give a significant reduction of the sebum surface percentage and the number of active pores. The placebo had no significant effect on any of the 3 read-outs.

### Example 7: Effect of Tetraselmis suecica Extract (Dried) on the Gene Expression of Human Keratinocytes

Neonatale humane epidermal keratinocytes (nHEK) were cultivated in EpiLife medium (Gibco) including HKGS-Kit (Gibco) at 5% CO₂ at 37° C. according to the supplier instructions.

The cells were treated for 24 hours with Tetraselmis suecica water extract obtained according to Example 1 by extracting at 80° C. at 0.025% or medium as vehicle control. Genomic target expression levels in extract treated cells were measured by RT-qPCR comparing to medium treatment.

RNA isolation took place using RNeasy® Mini Kit, Qiagen. Total RNA concentrations were measured using μCuvetteG 1.0 and BioPhotometer, Eppendorf by measuring the absorption at 260 nm. Purity control values, like E260/280 and E 260/230 were calculated simultaneously. Reverse transcription was done using high capacity RNA-to-cDNA Kit, Applied Biosystems, according to the supplier instructions. Samples were treated in the PCR Thermocycler, Biometra.

For the fast real-time PCR, cDNA was diluted with RNase-free water and TaqMan™ Fast Universal PCR Master Mix, Applied biosystems. Quantitative Real-Time PCR was done using StepOne Plus Fast Real Time PCR Instrument, Applied biosystems. Analysis was done with StepOne-Software and 2-ΔCT Method (normalized to endogenous control HTRP1 expression).

For upregulations RQ values≥2.0 and for downregulations RQ values<0.5 are considered to be relevant.

TABLE 7a
Modulation of gene expression of human epidermal
keratinocytes after treatment with 0.025 wt. % Tetraselmis suecica
water extract (prepared by re-dissolving the dried extract)

| | Indicated relevance but not | RQ |
|---|---|---|
| Genes | limited to | value |
| AQP3 [Aquaporin 3] | water/glycerol-transporting | 4.3 |
| | protein | |
| KRT1 [Keratin 1] | Differentiation | 12.8 |
| KRT10 [Keratin 10] | Differentiation | 14.2 |
| FLG [(Pro)Filaggrin] | Differentiation | 2.3 |
| FLG2 [Filaggrin Family Member 2] | Differentiation | 4.3 |
| SBSN [Suprabasin] | Differentiation | 7.3 |
| CSP14 [Caspase 14] | Differentiation | 10.9 |
| DMKN [Dermokine] | Differentiation | 10.6 |
| LIPN [Lipase Family Member N] | Differentiation | 14.3 |
| TGM5 [Transglutaminase 5] | Differentiation | 5.0 |
| S100A10 [S100 Calcium Binding | Differentiation | 3.2 |
| Protein A10] | | |
| S100A11 [S100 Calcium Binding | Differentiation | 3.2 |
| Protein A11] | | |
| S100A7 [S100 Calcium Binding | Differentiation/antimicrobial | 6.5 |
| Protein A7] | peptide | |
| S100A8 [S100 Calcium Binding | Differentiation/antimicrobial | 5.7 |
| Protein A8] | peptide | |
| S100A9 [S100 Calcium Binding | Differentiation/antimicrobial | 8.2 |
| Protein A9] | peptide | |
| SPRR1A [Small Proline Rich | Late differentiation | 6.5 |
| Protein 1A] | | |
| SPRR1B [Small Proline Rich | Late differentiation | 8.5 |
| Protein 1B] | | |
| SPRR2C [Small Proline Rich | Late differentiation | 2.6 |
| Protein 2C] | | |
| SPRR4 [Small Proline Rich Protein | Late differentiation | 2.9 |
| 4] | | |
| DSG1 [Desmoglein-1] | Desmosomes | 13.4 |
| DSG3 [Desmoglein-3] | Desmosomes | 5.1 |
| DSC1 [Desmocollin 1] | Desmosomes | 10.8 |
| DSP [Desmoplakin] | Desmosomes | 3.8 |
| EVPL [Enyoplakin] | Desmosomes | 4.5 |
| PKP1 [Plakophilin 1] | Desmosomes | 4.2 |
| CDH1 [Cadherin-1] | Adherens junctions | 3.0 |
| CTNNB1 [Catenin Beta 1] | Adherens junctions | 2.5 |
| CSTA [Cytostain A] | Desmosome stability | 2.9 |
| CLDN1 [Claudin 1] | Tight junctions | 7.6 |
| CLDN7 [Claudin 7] | Tight junctions | 6.2 |
| OCLN [Occludin] | Tight junctions | 7.5 |
| CGN [Cingulin] | Tight junctions | 8.5 |
| TJP1 [Tight junction protein ZO-1] | Tight junctions | 3.0 |
| F11R [F11 Receptor] | Tight junctions | 3.1 |
| F11R [F11 Receptor] | Tight junctions | 3.1 |
| GJA1 [Gap Junction Protein Alpha | Gap junctions | 2.9 |
| 1] | | |
| CXCR1 [C-X-C Motif Chemokine | Re-epithelialization | 6.9 |
| Receptor 1] | | |
| Serpine1 | Re-epithelialization | 3.2 |

The results clearly show that Tetraselmis extract surprisingly upregulates many genes involved in epidermal junctions, such as desmosomal (“mechanical”), tight, adherens and gap junctions relevant for cell-to-cell adhesion and allowance of the exchange of ions, second messengers, and small metabolites between adjacent cells in skin cells. These adhesion structures are essential not only for the maintenance of cell structure and integrity, but also for tissue development and morphogenesis. Mutations within the desmosome are e.g. the underlying cause of many skin fragility disorders.

Furthermore, genes relevant for differentiation, re-epithelialization and water/glycerol-transport are modulated by treatment with Tetraselmis extract.

In another experiment, the cells were treated for 24 hours with Tetraselmis suecica water dry extract obtained according to Example 1 from a different microalgae biomass batch either by extracting at 80° C. or at room temperature (18-23° C.) at 0.025% or medium as vehicle control. Genomic target expression levels of selected genes in extract treated cells were measured by RT-qPCR comparing to medium treatment as described above.

TABLE 7b
Modulation of gene expression of human epidermal keratinocytes
after treatment with 0.025% Tetraselmis suecica water extract
(prepared by re-dissolving the dried extract)

| | RQ value | |
|---|---|---|
| Genes | Extract 80° C. | Extract Room temp. |
| KRT1 [Keratin 1] | 8.0 | 1.0 |
| KRT10 [Keratin 10] | 7.9 | 1.0 |
| CSP14 [Caspase 14] | 4.0 | 1.0 |
| SPRR1A [Small Proline | 4.0 | 2.0 |
| Rich Protein 1A] | | |
| SPRR1B [Small Proline | 4.0 | 4.0 |
| Rich Protein 1B] | | |
| DSG1 [Desmoglein-1] | 8.0 | 2.0 |
| DSC1 [Desmocollin 1] | 4.0 | 1.0 |
| DSP [Desmoplakin] | 2.0 | 1.0 |
| CTNNB1 [Catenin Beta 1] | 2.0 | 1.0 |
| CLDN1 [Claudin 1] | 4.0 | 2.0 |
| OCLN [Occludin] | 4.0 | 4.1 |
| CGN [Cingulin] | 4.0 | 4.0 |

Results show that 6 genes (KRT1, KRT10, CSP14, DCS1, DSP, CTNNB1) were upregulated by the extract prepared at 80° C. whereas the extract prepared at room temperature had no effect. 6 of the selected genes (SPRRA1, SPRR1B, DSG1, CLDN1, OCLN, CGN) were upregulated by both extract.

### Example 8: Effect of Tetraselmis suecica Extract (Dried) on the Gene Expression of AMPs

HaCaT keratinocytes were cultivated in EpiLife medium (Gibco) at 5% CO₂ at 37° C.

The cells were treated for 24 hours with Tetraselmis suecica water dry extract obtained according to Example 1 by extracting at 80° C. at 0.05% or medium as vehicle control. Genomic target expression levels in extract treated cells were measured by RT-qPCR comparing to medium treatment.

RNA isolation took place using RNeasy® Mini Kit, Qiagen. Total RNA concentrations were measured using μCuvetteG 1.0 and BioPhotometer, Eppendorf by measuring the absorption at 260 nm. Purity control values, like E260/280 and E 260/230 were calculated simultaneously. Reverse transcription was done using RNA-to-cDNA Kit, Applied Biosystems, according to the supplier instructions. Samples were treated in the PCR Thermocycler, Biometra.

For the fast real-time PCR, cDNA was diluted with RNase-free water and TaqMan™ Fast Universal PCR Master Mix, Applied biosystems. Quantitative Real-Time PCR was done using StepOne Plus Fast Real Time PCR Instrument, Applied biosystems. Analysis was done with StepOne-Software and 2-ΔCT Method (normalized to endogenous control HTRP1 expression).

For upregulations RQ values≥2.5 and for downregulations RQ values<0.5 are considered to be relevant.

TABLE 8
Modulation of gene expression of AMPs in HaCaT keratinocytes
after treatment with 0.05% Tetraselmis suecica water dry extract

| Genes | RQ value |
|---|---|
| DEFB1 [beta-Defensin 1] | 20.8 |
| DEFB103A; DEFB103B [beta-Defensin 103 A/ | 3.1 |
| 103B] | |
| ADM [Adrenomedulin] | 3.1 |
| S100A7 [Psoriasin, S100 calcium binding | 7.4 |
| protein A7] | |

The results clearly show that Tetraselmis extract surprisingly also upregulates the gene expression of antimicrobial peptides such as beta-defensins, adrenomedullin and psoriasin in skin cells.

### Example 9: Cyclooxygenase (COX)-2 Inhibition Assay

COX-2 is the inducible rate limiting enzyme for prostaglandin, e.g. PGE2, synthesis. COX-2/PGE2 are expressed by keratinocytes and sebocytes. The test substance, Tetraselmis suecica water dry extract, is dissolved in assay buffer (Tris-HCl pH 8.0, 100 mM) and is given into a 96-well half area microplate. The co-enzyme HEME, the fluorometric substrate 10-acetyl-3,7-dihydroxy-phenoxanin (ADHP) and COX-2 are added. The half area microplate is incubated for 2 minutes at 600 rpm on a microplate shaker. Afterwards the substrate arachidonic acid is added. COX-2 converts arachidonic acid into the prostaglandin endoperoxide G2 (PGG2). PGG2 is reduced to the corresponding alcohol PGH2. During this reaction ADHP results in fluorescent resorufin. Resorufin is quantified at an extinction wavelength of 535 nm and an emission wavelength of 590 nm.

The inhibition of COX-2 activity in the presence of test substances was calculated according to the following equation:

$$Inhibition of COX \text{-} 2 [ \% ] = 100 - ( \frac{\begin{matrix}Resorufin test substance - \\ Resorufin control without COX \text{-} 2\end{matrix}}{\begin{matrix}Resorufin control - \\ Resorufin control without COX \text{-} 2\end{matrix}} \times 100 )$$

The abbreviations have the following meanings:

Resorufin test substance:

Resorufin concentration of the wells with test substance and with COX-2

Resorufin control:

Resorufin concentration of the wells without test substance, but with COX-2

Resorufin control without COX-2:

Resorufin concentration of the wells without test substance and without COX-2
Results are mean values from at least 2 independent experiments.

TABLE 9
COX-2 Inhibition by Tetraselmis suecica water extract (dried)

| Test concentration | COX-2 inhibition versus control |
|---|---|
| 0.025% | 34 ± 5% |

The results show that Tetraselmis extract also inhibits COX-2 enzyme activity.

### Example 10: Ex Vivo Human Skin—Filaggrin

Skin samples of approx. 8×3 mm (Ø×thickness) were cultured in an air-liquid interface in a perforated ring of stainless steel in contact with culture medium (modified Williams' E medium) up to day 6. After 24 acclimation, Tetraselmis suecica water dry extract obtained according to Example 1 by extracting at 80° C. at 0.3 ppm or medium as vehicle control were topically applied on the human skin explants (6 explants per each treatment). At day 6 of culture, the skin samples were embedded in an appropriate medium and frozen in liquid nitrogen. Quantitative analysis of filaggrin was performed on cryostat sections submitted to specific immunofluorescence staining. Twelve skin sections per treatment were immunostained with the selected antibody (filaggrin: rabbit polyclonal, SantaCruz). Each section was photographed by using a fluorescence microscope and the resulting image was analyzed. The amount of antigen present in each slide was evaluated by evaluating the intensity and the distribution of the stain within the epidermis (without stratum corneum). The obtained data was then normalized upon the length of the basal lamina.

TABLE 10
ex vivo human skin-filaggrin modulation

| Filaggrin | Control/ | Tetraselmis | Stimulation vs |
|---|---|---|---|
| score | vehicle | extract | control |
| Mean value | 40 | 107 | 163% |
| SEM | 7 | 15 | |

The results clearly show, that Tetraselmis suecica extract increases the epidermal filaggrin level of ex vivo human skin after topically application.

### Example 11: Ex Vivo Human Skin—Particle Matter (PM) Induced Barrier Damage

To evaluate the protective effect of Tetraselmis suecica water extract (dried) on cutaneous barrier damages induced by diesel particulate matter, ex vivo human skin explants used. Standard Reference Material® 1650b was obtained from the US National Institute of Standards and Technology (NIST) and is intended for use in evaluating analytical methods for the determination of selected polycyclic aromatic hydrocarbons (PAHs) and nitro-substituted PAHs (nitro-PAHs) in diesel particulate matter and similar matrices. It was collected from the heat exchangers of a dilution tube facility following 200 engine hours of particulate accumulation. Several direct injection four-cycle diesel engines, operating under a variety of conditions were used to generate this particulate material and it should be representative of heavy-duty diesel engine particulate emissions. Skin samples of approx. 8×3 mm (Ø×thickness) were cultured in a perforated ring of stainless steel in contact with culture medium (modified Williams' E medium) at environmental humidity up to day 3. 1650b was suspended in PBS. Hydrodispersion gels without (placebo) and with 0.01 and 0.05% Tetraselmis suecica water dry extract were formulated (Table 12). After 24 h acclimation, the formulations were topically applied on the skin explants before the application of the particulate matter 1650b outside the incubator for 90 min in order to let them dry. Afterwards the diesel particulate was topically applied at 10 μg/cm2 on the treated and untreated skin. Application of formulations and treatment with 1650b was renewed every day.

On day 3, the skin explants were harvested to evaluate skin barrier properties. Rhodamine B cannot penetrate the intact skin, thus the more rhodamine B is detectable inside the epidermis the more damaged the skin barrier is. Therefore, skin explants were stained with rhodamine B, cryo-fixed and cut at the cryostat for consequent image acquisition and analysis. The analysis of rhodamine B fluorescence was performed within the epidermis area. For each skin explant two sections were taken and fluorescent images acquired. For each image the upper dermis was analyzed by evaluating the fluorescence through Image-J application (NIH, USA). The obtained value was then normalized upon the dimension of the selected area.

TABLE 11
Formulation

| | | | 0.01% | 0.05% |
|---|---|---|---|---|
| | | | Tetra- | Tetra- |
| | | | selmis | selmis |
| Raw material | INCI | Placebo | extract | extract |
| Water | Water (Aqua) | Ad 100 | Ad 100 | Ad 100 |
| Hydrolite-5 | Pentyleneglycol | 2 | 2 | 2 |
| PCL liquid 100 | Cetearyl Octanoate | 3 | 3 | 3 |
| Lanette O | Cetearyl Alcohol | 2 | 2 | 2 |
| Mineral Oil 5°E | Mineral Oil | 3 | 3 | 3 |
| Eutanol G | Octyldodecanol | 4 | 4 | 4 |
| Abil 350 | Dimethicone | 0.5 | 0.5 | 0.5 |
| Pemulen TR1 | Acrylates/C10-30 Alkyl | 0.2 | 0.2 | 0.2 |
| | Acrylate Crosspolymer | | | |
| Ultrez-21 | Acrylates/C10-30 Alkyl | 0.05 | 0.05 | 0.05 |
| | Acrylate Crosspolymer | | | |
| Sodium | Sodium Hydroxide | 0.50 | 0.5 | 0.5 |
| hydroxide Sol. | | | | |
| 10% | | | | |
| Tetraselmis | Tetraselmis suecica extract | — | 0.01 | 0.05 |
| suecica extract | | | | |
| pH value | | 5.5 | | |

TABLE 12
Ex vivo human skin results-rhodamine B penetration

| | | | | 0.01% | 0.05% |
|---|---|---|---|---|---|
| | | | | Tetra- | Tetra- |
| Rhodamine B | | | | selmis | selmis |
| penetration | | | Placebo + | extract + | extract + |
| [L*/pixel] | Untreated | 1650b | 1650b | 1650b | 1650b |
| Mean | 0.244 | 0.934 | 0.798 | 0.650 | 0.516 |
| SEM | 0.034 | 0.151 | 0.149 | 0.069 | 0.074 |
| Stimulation versus | — | 283 | | | |
| untreated [%] | | | | | |
| Reduction versus | — | — | 15 | 30 | 45 |
| 1650b [%] | | | | | |
| Reduction versus | — | — | — | 19 | 35 |
| placebo [%] | | | | | |

As expected, topical treatment of human skin explants with particulate matter 1650b significantly increased the penetration of rhodamine B into the skin and thus cutaneous barrier damage.

The placebo led to a reduction of rhodamine B penetration by 15% versus 1650b treatment alone. Tetraselmis suecica extract led to a dose dependent reduction of rhodamine B penetration of 30 and 45% versus 1650b treatment alone and of 19 and 35% versus placebo+1650b treatment.

### Example 12: TEER Assay

Transepithelial electrical resistance (TEER) is a widely accepted quantitative technique to measure the integrity of tight junction dynamics in cell culture models of epithelial monolayers. TEER values are strong indicators of the integrity or strength of the cellular barriers. Increased resistance of a tissue is a result of higher density. Therefore, increased resistance relates to an improved skin barrier.

Neonatal humane epidermal keratinocytes (nHEKs) were seeded in a concentration of 1.5×10⁵ cells per inserts in 0.47 cm² cell culture inserts. After incubation with cell culture medium for four days, Tetraselmis suecica extract prepared according to the description given in Example 1 was systemically applied for eight days within the cell culture media in a final volume as listed below. Following the substance treatment, the TEER was determined. Cell culture medium was used as control.

TABLE 13
TEER essay results

| | | Medium | Medium | 0.025% |
|---|---|---|---|---|
| | | control | control | Tetraselmis |
| Readout | Blank* | start | end | suecica extract |
| Readings | 31 | 42 | 36 | 66 |
| | | 45 | 37 | 69 |
| | | 42 | 37 | 75 |
| Mean value | 31 | 43 | 36.67 | 70 |
| Standard | | 1.73 | 0.58 | 4.58 |
| deviation | | | | |
| TEER [Ω cm²] | 14.57 | 20.21 | 17.23 | 32.90 |

*Insert without cells

The results clearly show, that Tetraselmis suecica extract increased the TEER value when compared to untreated (medium control).

### Example 13: Formulation Examples

In formulations 1-22 the following two perfume oils PFO1 and PFO2 were each used as fragrance (DPG=dipropylene glycol).

TABLE 14
Perfume oil PFO1 with rose smell (amounts in parts b.w.)

| Component | Amount |
|---|---|
| Acetophenone, 10% in DPG | 10.00 |
| n-Undecanal | 5.00 |
| Aldehyde C14, so-called (peach aldehyde) | 15.00 |
| Allylamyl glycolate, 10% in DPG | 20.00 |
| Amyl salicylate | 25.00 |
| Benzyl acetate | 60.00 |
| Citronellol | 80.00 |
| d-Limonene | 50.00 |
| Decenol trans-9 | 15.00 |
| Dihydromyrcenol | 50.00 |
| Dimethylbenzylcarbinyl acetate | 30.00 |
| Diphenyloxide | 5.00 |
| Eucalyptol | 10.00 |
| Geraniol | 40.00 |
| Nerol | 20.00 |
| Geranium oil | 15.00 |
| Hexenol cis-3, 10% in DPG | 5.00 |
| Hexenyl salicylate cis-3 | 20.00 |
| Indole, 10% in DPG | 10.00 |
| Alpha-ionone | 15.00 |
| Beta-ionone | 5.00 |
| Lilial ® (2-methyl-3-(4-tert-butyl-phenyl)propanal) | 60.00 |
| Linalool | 40.00 |
| Methylphenyl acetate | 10.00 |
| Phenylethyl alcohol | 275.00 |
| Styrolyl acetate | 20.00 |
| Terpineol | 30.00 |
| Tetrahydrolinalool | 50.00 |
| Cinnamyl alcohol | 10.00 |
| Total: | 1,000.00 |

TABLE 15
Perfume oil PFO2 with white blossom and musk
smell (amounts in parts b.w.)

| Component | Amount |
|---|---|
| Benzyl acetate | 60.00 |
| Citronellyl acetate | 60.00 |
| Cyclamenaldehyde (2-methyl-3-(4-isopropylphenyl) | 20.00 |
| propanal | |
| Dipropylene glycol (DPG) | 60.00 |
| Ethyllinalool | 40.00 |
| Florol (2-isobutyl-4-methyltetrahydro-2H-pyran-4-ol) | 30.00 |
| Globanone ® [(E/Z)-8-cyclohexadecen-1-one] | 180.00 |
| Hedione ® (methyldihydrojasmonate) | 140.00 |
| Hexenyl salicylate, cis-3 | 10.00 |
| Vertocitral (2,4-dimethyl-3-cyclohexenecarboxaldehyde) | 5.00 |
| Hydratropaaldehyde, 10% in DPG | 5.00 |
| Isodamascone (1-(2,4,4-trimethyl-2-cyclohexen- | 5.00 |
| 1-yl)-2-buten-1-one, 10% in DPG | |
| Isomuscone (cyclohexadecanone) | 40.00 |
| Jacinthaflor (2-methyl-4-phenyl-1,3-dioxolane) | 10.00 |
| Cis-jasmone, 10% in DPG | 20.00 |
| Linalool | 50.00 |
| Linalyl acetate | 30.00 |
| Methyl benzoate, 10% in DPG | 25.00 |
| para-Methyl cresol, 10% in DPG | 10.00 |
| Nerol | 20.00 |
| Phenylpropylaldehyde | 5.00 |
| 2-Phenylethyl alcohol | 82.00 |
| Tetrahydrogeraniol | 13.00 |
| 2,2-Dimethyl-3-cyclohexyl-1-propanol | 80.00 |
| Total: | 1,000.00 |

TABLE 16
Cosmetic formulations (amounts in parts b.w.)

| Ingredients | 1 | 2 | 3 | 4 | 5 | 6 | 7 | 8 | 9 | 10 | 11 |
|---|---|---|---|---|---|---|---|---|---|---|---|
| Tetraselmis suecica extract 2.5% in glycerin/water | 2 | 1 | | | 5 | 1 | | | 0.4 | 1.5 | 3 |
| Water, Glycerin, Tetraselmis suecica extract | | | | | | | | | | | |
| Spray-dried Tetraselmis suecica extract containing | | | 1 | 0.5 | | | 1 | 0.2 | | | |
| 95% maltodextrin, 5% extract matter | | | | | | | | | | | |
| Maltodextrin, Tetraselmis suecica extract | | | | | | | | | | | |
| Actipone Alpha-Pulp | 0.1 | | | | | | | | | 1 | |
| Aqua, Butylene Glycol, Malic Acid, Actinidia | | | | | | | | | | | |
| Chinensis Fruit Extract, Citrus Aurantium Dulcis | | | | | | | | | | | |
| Juice, Citrus Paradisi Juice, Pyrus Malus Juice, | | | | | | | | | | | |
| Trideceth-9, Prunus Amydalus Dulcis Seed Extract | | | | | | | | | | | |
| Allantoin | 0.1 | | | | | | | | | | |
| Allantoin | | | | | | | | | | | |
| Aloe Vera Gel Conc. 10:1 | | | | | | 0.2 | | | | | |
| Aloe Barbadensis(Aloe) Leaf Juice | | | | | | | | | | | |
| Aluminium Stearate | | | | 1.2 | | | | | | | |
| Aluminium Stearate | | | | | | | | | | | |
| Asebiol | | | | | | | 3 | | | | |
| Water, Pyridoxine HCL, Niacinamide, Glycerin, | | | | | | | | | | | |
| Panthenol, Hydrolyzed Yeast Protein, Threonine, | | | | | | | | | | | |
| Allantoin, Biotin | | | | | | | | | | | |
| Beta-Arbutin | | | | | | | | | 1 | | |
| Arbutin | | | | | | | | | | | |
| Arlypon ® F | | | | | | | 2 | | | | |
| Laureth-2 | | | | | | | | | | | |
| Asensa ® SC 220 | | | | | | | | | | | 2 |
| Polyethylene | | | | | | | | | | | |
| Azelaic acid | | | | | | | | 0.5 | | | |
| Azelaic acid | | | | | | | | | | | |
| Biotive L-Arginine | | 0.6 | | | | | | | | | |
| Arginine | | | | | | | | | | | |
| Biotive Troxerutin | | 0.5 | | | | | | | | | |
| Troxerutin | | | | | | | | | | | |
| (—)-alpha-Bisabolol | | | | 0.1 | | | | | | | |
| Bisabolol | | | | | | | | | | | |
| Carbopol Aqua SF-1 Polymer | | | | | 5 | | | | | | |
| Acrylates Copolymer | | | | | | | | | | | |
| Carbopol ® Ultrez-10 | | 0.2 | 0.2 | | | | | | 0.2 | 0.3 | |
| Carbomer | | | | | | | | | | | |
| Citric acid 10% in water | | | | | 0.2 | | 0.5 | | | | |
| Citric acid, water | | | | | | | | | | | |
| Colour | | | 0.04 | | | | | | | | |
| Crinipan ® AD | | | | | | | 0.3 | | | | |
| Climbazole | | | | | | | | | | | |
| Cutina ® AGS | | | | | | | | | | | 1.5 |
| Glycol Distearate | | | | | | | | | | | |
| Cutina ® PES | | 2 | | | | | | | | | |
| Pentaerythrityl Distearate | | | | | | | | | | | |
| D-Panthenol | | | | | | | 0.5 | | | | |
| Panthenol | | | | | | | | | | | |
| Dehyton K | | | | | 8 | | 8 | | | | |
| Cocamidopropyl Betaine | | | | | | | | | | | |
| Dow Corning 200(100cs) | 2 | 2 | | | | | | | 0.5 | | |
| Silicone Fluid Dimethicone | | | | | | | | | | | |
| Dracorin ® CE | | | 5 | | | | | | | 2.5 | |
| Glyceryl Stearate Citrate | | | | | | | | | | | |
| Dracorin GOC | | | | | | | | 2.5 | | | |
| Glyceryl Oleate Citrate, Caprylic/Capric Triglyceride | | | | | | | | | | | |
| Dragocalm ® | 1 | | | | | | | | | | |
| Water (Aqua), Glycerin, Avena Sativa (Oat) Kernel Extract | | | | | | | | | | | |
| Dragoderm ® | | | | | | | 0.5 | | | | |
| Glycerin, Triticum Vulgare (Wheat) Gluten, Water (Aqua) | | | | | | | | | | | |
| Dragosan ® W/O P | | | | 8 | | | | | | | |
| Sorbitan Isostearate, Hydrogenated Castor Oil, Ceresin, | | | | | | | | | | | |
| Beeswax (Cera Alba) | | | | | | | | | | | |
| Dragosantol ® 100 | | | | | 0.2 | | | | | | |
| Bisabolol | | | | | | | | | | | |
| Dragosine ® | | 0.2 | 0.2 | | | | | | | | |
| Carnosine | | | | | | | | | | | |
| Dragoxat ® 89 | | 5 | | 7 | | | | 1 | | 5 | |
| Ethylhexyl Isononanoate | | | | | | | | | | | |
| Disodium EDTA | 0.1 | 0.1 | 0.1 | | | | | 0.1 | 0.1 | 0.05 | 0.05 |
| Disodium EDTA | | | | | | | | | | | |
| Emulsiphos ® | | 2 | | | | | | | 1.5 | | |
| Potassium Cetyl Phosphate, Hydrogenated Palm Glycerides | | | | | | | | | | | |
| Estearina L2SM GS | | | | | | | | | | | 2 |
| Stearic Acid, Palmitic Acid | | | | | | | | | | | |
| Ethanol | | | | | | 2 | | | | | |
| Ethanol | | | | | | | | | | | |
| Extrapone ® Aloe vera | | | | 1 | | | | | | | |
| Water (Aqua), Aloe Barbadensis, Propylene Glycol, Alcohol | | | | | | | | | | | |
| Extrapone Eucalyptus | | | | | | | | | | | 1 |
| Aqua, Propylene Glycol, Eucalyptus Globulus Leaf Extract | | | | | | | | | | | |
| Extrapone Iris B | | | | | | 0.5 | | | | | |
| Aqua, Propylene Glycol, PEG-40 Hydrogeanted Castor Oil, | | | | | | | | | | | |
| Trideceth-9, Bisabolol, Iris Germanica Root Extract | | | | | | | | | | | |
| Extrapone ® Witch Hazel | 1 | | | | | | | | | | |
| Propylene Glycol, Hamamelis Virginiana (Witch Hazel) | | | | | | | | | | | |
| Water, Water (Aqua), Hamamelis Virginiana (Witch Hazel) Extract | | | | | | | | | | | |
| Food Color Brown E172 + E171 Powder | | 2 | | | | | | 1.5 | | | |
| Titanium Dioxides (CI77891), Iron Oxides (CI77492), Iron | | | | | | | | | | | |
| Oxides (CI77491), Iron Oxides (CI77499) | | | | | | | | | | | |
| Food Color Titanium Dioxide Powder E171 | | | | | | | | | | | 3 |
| Titanium Dioxides (CI77891) | | | | | | | | | | | |
| Frescolat ® MGA | | | | | | | | | | | 0.5 |
| Menthone Glycerin Acetal | | | | | | | | | | | |
| Frescolat ® ML | | | | | | 0.3 | 0.2 | | | 0.3 | |
| Menthyl Lactate | | | | | | | | | | | |
| Frescolat Plus | | | 0.2 | | | | | | | | |
| Menthol, Menthyl Lactate | | | | | | | | | | | |
| Frescolat ® X-Cool | | | | | 0.2 | | | | | | |
| Menthyl Ethylamido Oxalate | | | | | | | | | | | |
| Genapol ® LRO Liquid | | | | | | | 37 | | | | |
| Sodium Laureth Sulfate | | | | | | | | | | | |
| Glycerin | | | 3 | 3 | | | | 4.5 | 3 | 1.5 | 3 |
| Glycerin | | | | | | | | | | | |
| Hydrolite ® 5 | 3 | | | | 2 | | | | | | |
| Pentylene Glycol | | | | | | | | | | | |
| Hydroviton-24 ® | | | | 1 | | | | | | | |
| Water (Aqua), Pentylene Glycol, Glycerin, Lactic Acid, Sodium | | | | | | | | | | | |
| Lactate, Serine, Urea, Sorbitol, Sodium Chloride, Allantoin | | | | | | | | | | | |
| Hydroviton ® Plus 2290 | | | 2 | | | | | | | | |
| Water (Aqua), Pentylene Glycol, Glycerin, Fructose, Urea, Citric | | | | | | | | | | | |
| acid, Sodium Hydroxide, Maltose, Sodium PCA, Sodium Chloride, | | | | | | | | | | | |
| Sodium Lactate, Trehalose, Allantoin, Sodium Hyaluronate, Glucose | | | | | | | | | | | |
| Isoadipate | | | 2 | | | | | | 2 | | |
| Diisopropyl Adipate | | | | | | | | | | | |
| Isodragol ® | 1 | | | | | | | | | | |
| Triisononanoin | | | | | | | | | | | |
| Jojoba Oil | | | | 0.3 | | | | | | | |
| Simmondsia Chinensis (Jojoba) Seed Oil | | | | | | | | | | | |
| Kaolin | | | | | | | | | | | 10 |
| Kaolin | | | | | | | | | | | |
| Keltrol ® CG-RD | | 0.2 | 0.1 | | | | | 0.3 | 0.2 | 0.3 | 1.2 |
| Xanthan Gum | | | | | | | | | | | |
| Kojic acid | | | | | | | | | 0.5 | | |
| Kojic Acid | | | | | | | | | | | |
| KP-545 | | | | | | | | 1 | | | |
| Cyclopentasiloxane Acrylates/Dimethicone Copolymer | | | | | | | | | | | |
| Lanette ® 16 | | | | | | | | | 1.5 | 2 | |
| Cetyl Alcohol | | | | | | | | | | | |
| Lanette ® 22 | | | | | | | | | | 3 | |
| Behenyl Alcohol | | | | | | | | | | | |
| Lanette ® O | | | 5 | | | | | | | 2 | |
| Cetearyl Alcohol | | | | | | | | | | | |
| Magnesium Sulfate | | | | 0.7 | | | | | | | |
| Magnesium Sulfate | | | | | | | | | | | |
| Mineral Oil | | | | 5 | | | | | | | |
| Paraffinum Liquidum | | | | | | | | | | | |
| Neo Heliopan ® 303 | | 4 | | | | | | 10 | | | |
| Octocrylene | | | | | | | | | | | |
| Neo Heliopan ® 357 | | 2 | | | | | | 4 | 2 | | |
| Butylmethoxydibenzoyl-methane | | | | | | | | | | | |
| Neo Heliopan ® AP 15% Lösung, neutralisiert mit L-Arginin | | 6.7 | | | | | | | | | |
| Aqua, Disodium Phenyl Dibenzimidazole Tetrasulfonate, Arginin | | | | | | | | | | | |
| Neo Heliopan ® AV | | | | | | | | | 7.5 | | |
| Ethylhexyl Methoxycinnamate | | | | | | | | | | | |
| Neo Heliopan ® BB | | | | | | | | | 3 | | |
| Benzophenone-3 | | | | | | | | | | | |
| Neo Heliopan ® E 1000 | | 1 | | | | | | | | | |
| Isoamyl p.Methoxycinnamate | | | | | | | | | | | |
| Neo Heliopan ® HMS | | | | | | | | 7 | 10 | | |
| Homosalate | | | | | | | | | | | |
| Neo Heliopan ® OS | | 3 | | | | | | 5 | 5 | | |
| Ethylhexyl Salicylate | | | | | | | | | | | |
| Neo Heliopan ® Hydro 20% Lösung, neutralisiert mit | | 10 | | | | | | 3.5 | | | |
| Biotive Arginine | | | | | | | | | | | |
| Aqua, Phenylbenzimidazole, Sulphonic Acid, Arginin | | | | | | | | | | | |
| Neo-PCL Water Soluble N | | | | | | | 1.5 | | | | 2 |
| Trideceth-9, PEG-5 Ethylhexanoate, Water (Aqua) | | | | | | | | | | | |
| Neutral oil | | | 2 | | | | | | | | |
| Caprylic/Capric Triglyceride | | | | | | | | | | | |
| Niacinamide | | | | 0.5 | 3 | 0.3 | | | | | 0.75 |
| Niacinamide | | | | | | | | | | | |
| Ozokerite Wax 2389 | | | | 2 | | | | | | | |
| Ozokerite | | | | | | | | | | | |
| Parfume oil PFO1 or PFO2 | 0.05 | 0.3 | 1 | 0.3 | | 0.3 | 0.5 | | 0.3 | 0.1 | 0.5 |
| Parfum | | | | | | | | | | | |
| Passion Fruit Oil | | | 1 | | | | | | | | |
| Refined Passiflora Edulis seed oil | | | | | | | | | | | |
| PCL-Liquid 100 | 3 | 2 | | 5 | | | | | | | |
| Cetearyl Ethylhexanoate | | | | | | | | | | | |
| PCL-Solid | 1 | | | | | | | | | 2 | |
| Stearyl Heptanoate, Stearyl Caprylate | | | | | | | | | | | |
| Pemulen ® TR-2 | 0.6 | | | | | | | 0.15 | | | |
| Acrylates/C10-30 Alkyl Acrylate Crosspolymer | | | | | | | | | | | |
| Phenethyl Alcohol | | | | | 0.2 | | | | | | |
| Phenethyl Alcohol | | | | | | | | | | | |
| Phytoconcentrole ® | 1 | | | | | | | | | | |
| Shea Butter, Glycine Soja (Soybean) Oil, Butyrospermum Parkii | | | | | | | | | | | |
| (Shea Butter) | | | | | | | | | | | |
| Plantacare PS 10 | | | | | | | | | | | 5 |
| Sodium Laureth Sulfate, Lauryl Glucoside | | | | | | | | | | | |
| Polymer JR 400 | | | | | | | 0.4 | | | | |
| Sodium Laureth Sulfate, Lauryl Glucoside | | | | | | | | | | | |
| Retinol | | | | 0.1 | | | | | | | |
| Retinol | | | | | | | | | | | |
| Salicylic acid | | | | | | | | | | 0.5 | 0.3 |
| Salicylic Acid | | | | | | | | | | | |
| Sodium Ascorbyl Phosphate | | | | | | | | | 1 | | |
| Sodium Ascorbyl Phosphate | | | | | | | | | | | |
| Sodium Chloride | | | | | | | 0.1 | | | | |
| Sodium Chloride | | | | | | | | | | | |
| Sodium Hydroxide 10% Solution | 1 | | 0.5 | | 2 | | | | 0.2 | 1.9 | 1.1 |
| Sodium Hydroxide 10% Solution | | | | | | | | | | | |
| Softisan 100 | | | 6 | | | | | | | | |
| Hydrogenated Coco-Glycerides | | | | | | | | | | | |
| Solubilizer | | | | | | 3 | | | | | |
| PEG-40 Hydrogenated Castor Oil, Trideceth-9, Propylene Glycol, | | | | | | | | | | | |
| Water (Aqua) | | | | | | | | | | | |
| Sulfetal LA | | | | | 12 | | | | | | |
| Ammonium Lauryl Sulfate | | | | | | | | | | | |
| SymCalmin ® | 1 | | | | | | 0.1 | | | | 0.5 |
| Butylene Glycol, Pentylene Glycol, Hydroxyphenyl | | | | | | | | | | | |
| Propamidobenzoic Acid | | | | | | | | | | | |
| SymClariol ® | | | 0.1 | | 1 | | | | 0.2 | 0.38 | |
| Decylene Glycol | | | | | | | | | | | |
| SymDecanox HA | | | 1 | 2 | | | | | | | |
| Caprylic/Capric Triglyceride, Hydroxymethoxyphenyl Decanone | | | | | | | | | | | |
| Symdiol ® 68 | 1 | 0.5 | 0.5 | | | | | 0.5 | | 0.8 | |
| 1,2-Hexanediol, Caprylyl Glycol | | | | | | | | | | | |
| SymFinity ® 1298 | | | | 0.05 | | | | | | | |
| Echinacea Purpurea Extract | | | | | | | | | | | |
| SymGlucan ® | 1 | | | | | | | 2 | | | |
| Water (Aqua), Glycerin, Beta-Glucan | | | | | | | | | | | |
| SymHair ® Force 1631 | | | | | | | 2 | | | | |
| Pentylene Glycol, Isochrysis galbana Extract | | | | | | | | | | | |
| SymHelios ® 1031 | | 0.3 | | | | | | | | | |
| Benzylidene Dimethoxydimethylindanone | | | | | | | | | | | |
| SymLift | | 2 | | | | | | | | | |
| Water, Trehalose, Glycerin, Pentylene glycol, beta-Glucan, | | | | | | | | | | | |
| Hordeum Vulgare Seed Extract, Sodium Hyaluronate, 1,2- | | | | | | | | | | | |
| Hexanediol, Caprylyl glycol, Sodium Benzoate, Maltodextrin | | | | | | | | | | | |
| SymMatrix | | 0.2 | | | | | | | | | |
| Maltodextrin, Rubus Fruticosus (Blackberry) Leaf Extract | | | | | | | | | | | |
| SymMollient S | | | 1 | | | | | | | | |
| Cetearyl Nonanoate | | | | | | | | | | | |
| SymMollient ® W/S | | | 1 | | 2 | 1.5 | 2 | | | | |
| Trideceth-9, PEG-5 Isononanoate, Water (Aqua) | | | | | | | | | | | |
| SymOcide ® C | | | | | | 0.1 | | | | | |
| o-Cymen-5-ol | | | | | | | | | | | |
| SymOcide ® PC | | | | | | | | | 1 | | |
| Phenoxyethanol, Caprylyl Glycol, | | | | | | | | | | | |
| SymOcide ® PH | | | | | | | 1 | | | | |
| Phenoxyethanol, Hydroxyacetophenone, Caprylyl Glycol, | | | | | | | | | | | |
| Water (Aqua) | | | | | | | | | | | |
| SymOcide ® PS | | | | | 0.8 | | | | | | 0.8 |
| Phenoxyethanol, Decylene Glycol, 1,2-Hexanediol | | | | | | | | | | | |
| SymOcide ® PT | | | | 0.8 | | | | | | | |
| Phenoxyethanol, Tropolone | | | | | | | | | | | |
| SymPeptide ® 225 | | | | 1 | | | | | | | |
| Glycerin, Water (Aqua), Myristoyl Pentapeptide-11 | | | | | | | | | | | |
| SymRelief ® 100 | | | | | | 0.1 | | | | | |
| Bisabolol, Zingiber Officinale (Ginger) Root Extract | | | | | | | | | | | |
| SymRelief ® S | | | | | | | | | | 0.1 | |
| Bisabolol, Hydroxymethoxyphenyl Decanone | | | | | | | | | | | |
| SymRepair ® 100 | | 1 | | | | | | | | | |
| Hexyldecanol, Bisabolol, Cetylhydroxyproline Palmitamide, | | | | | | | | | | | |
| Stearic Acid, Brassica Campestris (Rapeseed) Sterols | | | | | | | | | | | |
| SymSave ® H | | 0.5 | 0.5 | | | 0.5 | | 0.5 | | | |
| Hydoxyacetophenone | | | | | | | | | | | |
| SymSitive ® 1609 | 1 | | | | | | | | 0.5 | | |
| Pentylene Glycol, 4-t-Butylcyclohexanol | | | | | | | | | | | |
| SymVital ® AgeRepair 3040 | | 0.1 | | | | | | | | | |
| Zingiber Officinale (Ginger) Root Extract | | | | | | | | | | | |
| SymWhite ® 377 | | | | | | | | | 0.5 | | |
| Phenylethyl Resorcinol | | | | | | | | | | | |
| Vitacel CS 20 FC | | | | | | | | 3 | | | |
| Cellulose | | | | | | | | | | | |
| Vitamin A Palmitate | | | | 0.1 | | | | | | | |
| Retinyl Palmitate | | | | | | | | | | | |
| Vitamin E Acetate | | 0.5 | | 0.2 | | | | 0.5 | | 0.25 | |
| Tocopheryl Acetate | | | | | | | | | | | |
| Willow bark extract | | | | | | | | | | | 0.1 |
| Salix Alba Extract | | | | | | | | | | | |
| Xiameter PMX-345 | | | | | | | | | | 6 | |
| Cyclopentasiloxane, Cyclohexasiloxane | | | | | | | | | | | |
| Zetesol LA-2 | | | | | 26 | | | | | | |
| Ammonium Laureth Sulfate | | | | | | | | | | | |

| | |
|---|---|
| Water | Ad to 100 |

1 = Skin calming balm for sensitive oily skin
2 = Tinted Face Balm, SPF 15
3 = Rinse-off purifying mask for greasy skin
4 = Night cream W/O
5 = Facial Cleansing gel
6 = Face tonic for oily skin
7 = Anti-dandruff hair shampoo for greasy hair
8 = Sunscreen fluid for acne prone skin, SPF 30
9 = Skin lightening day care fluid O/W for impure oily skin
10 = Anti-acne skin cream
11 = 3 in 1 Skin purifying Wash + Scrub + Mask

TABLE 17
Cosmetic formulations 12 to 22 (amounts in parts b.w.)

| Ingredients | 12 | 13 | 14 | 15 | 16 | 17 | 18 | 19 | 20 | 21 | 22 |
|---|---|---|---|---|---|---|---|---|---|---|---|
| Tetraselmis suecica extract 2.5% in glycerin/water | 1.2 | 3 | | | 0.2 | 2 | | 0.4 | 1 | | 3 |
| Water, Glycerin, Tetraselmis suecica extract | | | | | | | | | | | |
| Spray-dried Tetraselmis suecica extract containing | | | 2 | 0.8 | | | 0.3 | | | 1 | |
| 95% maltodextrin, 5% extract matter | | | | | | | | | | | |
| Maltodextrin, Tetraselmis suecica extract | | | | | | | | | | | |
| Actipone ® White Tea GW | 1 | | | | | | | | | | |
| Aqua, Glycerin, Camellia Sinensis Leaf Extract | | | | | | | | | | | |
| Actipone ® Witch Hazel | | | | | | | 3 | | 1 | | |
| Hamamelis Virginiana Bark/Leaf/Twig Extract, Alcohol, | | | | | | | | | | | |
| Hamamelis Virginiana Water | | | | | | | | | | | |
| Actipone ® Black Currant GW | | | | | | 1 | | | | | |
| Aqua, Glycerin, Ribes Nigrum Juice | | | | | | | | | | | |
| Amisoft ® CS-11/CS-11(F) | | | | | 0.5 | | | | | | |
| Sodium Cocoyl Glutamate | | | | | | | | | | | |
| Andiroba Oil, refined | | | | | | | | | | | 0.3 |
| Carapa Guaianensis Seed Oil | | | | | | | | | | | |
| Aristoflex ® AVC | | | | | | | | | 0.5 | | |
| Ammonium Acryloyoldimethyltaurate/VP Copolymer | | | | | | | | | | | |
| 5-Alpha-Avocuta | | | | | | | | 1 | | | |
| Butyl Avocadate | | | | | | | | | | | |
| Beeswax | | | | | | | | 5 | | | |
| Cera Alba | | | | | | | | | | | |
| Butylene Glycol | | 0.5 | | | | | 5 | | | | |
| Butylene Glycol | | | | | | | | | | | |
| Candelilla Wax | | | | | | | | 15 | | | |
| Euphorbia Cera (Candelilla) Wax | | | | | | | | | | | |
| Carnauba Wax | | | | | | | | 5 | | | |
| Cera Carnaubae depurata | | | | | | | | | | | |
| Carbopol ® Aqua SF-1 Polymer | | | | | 10 | | | | | | |
| Acrylates Copolymer | | | | | | | | | | | |
| CeramideBio | | | | | | | | | 0.5 | | |
| Cetylhydroxyproline Palmitamide | | | | | | | | | | | |
| Citric acid 10% in water | | | 0.5 | | | | | | 0.2 | | |
| Citric acid, water | | | | | | | | | | | |
| Crinipan ® AD | | | | | | | | | | 0.2 | |
| Climbazole | | | | | | | | | | | |
| Disodium EDTA | 0.1 | 0.05 | | | 0.1 | | | | | | 0.1 |
| Disodium EDTA | | | | | | | | | | | |
| Dow Corning 345 Fluid | | | | | | | | 5 | | | |
| Cyclomethicone | | | | | | | | | | | |
| Dow Corning 556 Fluid | | | | | | | | 4 | | | |
| Phenyl Trimethicone | | | | | | | | | | | |
| Dow Corning 2502 Fluid | | | | | | | | 5 | | | |
| Cetyl Dimethicone | | | | | | | | | | | |
| D-Panthenol 75 L | | | | | | | | | | 0.3 | |
| Panthenol | | | | | | | | | | | |
| Dracorin GOC | 2.5 | | | | | | | | | | |
| Glyceryl Oleate Citrate, Caprylic/Capric Triglyceride | | | | | | | | | | | |
| Dragoxat ® 89 | 5 | | | 20 | | | | | 2 | | |
| Ethylhexyl Isononanoate | | | | | | | | | | | |
| Emulsiphos ® | | | | | | | | | 2 | | |
| Potassium Cetyl Phosphate, Hydrogenated Palm Glycerides | | | | | | | | | | | |
| Ethanol | | | | | | 5 | 5 | | | 10 | |
| Alcohol, Aqua | | | | | | | | | | | |
| Evermat | | | 3 | | | | | | | | |
| Enantia chlorantha bark extract | | | | | | | | | | | |
| Extrapone ® Strawberry B | | | | | | 1 | | | | | |
| Aqua, Propylene Glycol, Citric Acid, Trideceth-9, Bisabolol, | | | | | | | | | | | |
| Fragaria Ananassa Fruit Extract | | | | | | | | | | | |
| Extrapone ® Tiger Grass | | | | | | | 5 | | 1 | | |
| Aqua, Glycerin, PEG-40 Hydrogenated Castor Oil, Trideceth-9, | | | | | | | | | | | |
| Centella Asiatica Extract | | | | | | | | | | | |
| Flowerconcentrole ® | | | | | | | | | 2 | | |
| Frangipani Pentylene Glycol, Bisabolol, Plumeria Acutifolia | | | | | | | | | | | |
| Flower Extract L | | | | | | | | | | | |
| Frescolat ® ML | | | | | 0.3 | 0.3 | 0.3 | | | 0.5 | |
| Menthyl Lactate | | | | | | | | | | | |
| Glycerin | 3 | 3 | 3 | | | 5 | | | | | 1 |
| Glycerin | | | | | | | | | | | |
| Green Pigment | | | | | | | | 0.85 | | | |
| CI77288, Triethoxycaprylylsilane | | | | | | | | | | | |
| Hexylene Glycol | | | | 25 | | | | | | | |
| Hexylene Glycol | | | | | | | | | | | |
| Hispagel ® 200 | | | | | | 1 | 1 | | | | |
| Glycerin, Glyceryl Polyacrylate | | | | | | | | | | | |
| Hydrolite ® 5 | 1.5 | | | 3 | 1 | 4 | 5 | | | 7 | |
| Pentylene Glycol | | | | | | | | | | | |
| Hydrolite ® 6 | | | | | 0.5 | | | | | | |
| 1,2-Hexanediol | | | | | | | | | | | |
| Hydromoist ® L | | | | | | | | | | | 1 |
| Aqua, Hydrolyzed Lupine Seed Extract | | | | | | | | | | | |
| Hydroviton ® Plus 2290 | | | 1 | | | 1 | | | | | |
| Water (Aqua), Pentylene Glycol, Glycerin, Fructose, Urea, Citric | | | | | | | | | | | |
| acid, Sodium Hydroxide, Maltose, Sodium PCA, Sodium Chloride, | | | | | | | | | | | |
| Sodium Lactate, Trehalose, Allantoin, Sodium Hyaluronate, Glucose | | | | | | | | | | | |
| Icroquat Behenyl TMS-50 | | | | | | | | | | | 2 |
| Behentrimonium Methosulfate, Cetyl Alcohol, Butylene Glycol | | | | | | | | | | | |
| Isoadipate | | | | | | | | 12.7 | | | |
| Diisopropyl Adipate | | | | | | | | | | | |
| Isodragol ® | | | | | | | | 8 | | | |
| Triisononanoin | | | | | | | | | | | |
| Isopropyl Myristate | | | | | | | | | | | 2 |
| Isopropyl Myristate | | | | | | | | | | | |
| Jaguar ® Excel | | | | | | | | | 0.1 | | |
| Guar Hydroxypropyltrimonium Chloride | | | | | | | | | | | |
| Jojoba Oil | | | | | | | | 8 | | | 0.5 |
| Simmondsia Chinensis (Jojoba) Seed Oil | | | | | | | | | | | |
| Keltrol ® CG-T | | | | | | 0.1 | 0.2 | | | 0.3 | |
| Xanthan Gum | | | | | | | | | | | |
| Lactic acid | | | | 0.2 | | | | | | | |
| Lactic acid | | | | | | | | | | | |
| Lanette ® 16 | 1 | 1 | | | | | | | | | 3 |
| Cetyl Alcohol | | | | | | | | | | | |
| Lanette ® 18 | | | | | | | | | 4 | | |
| Stearyl Alcohol | | | | | | | | | | | |
| Lanette ® 22 | | | | | | | | | 2 | | |
| Behenyl Alcohol | | | | | | | | | | | |
| Lanette ® O | | | | | | | | | 1 | | 4.5 |
| Cetearyl Alcohol | | | | | | | | | | | |
| Medialan ® LD | | | 10 | | | | | | | | |
| Sodium Lauroyl Sarcosinate | | | | | | | | | | | |
| Mineral Oil | | | | | | | | | | | 1 |
| Paraffinum Liquidum | | | | | | | | | | | |
| Miniporyl ® | 1 | | | | | | | | | | |
| Isopentyldiol, Trifolium Pratense (Clover) Flower Extract | | | | | | | | | | | |
| Neo-PCL Water Soluble N | | | | | | | 1.5 | | | | |
| Trideceth-9, PEG-5 Ethylhexanoate, Aqua | | | | | | | | | | | |
| Niacinamide | | 3 | | 1 | 2 | | | | | | |
| Niacinamide | | | | | | | | | | | |
| Parfume oil PFO1 or PFO2 | 0.5 | | 0.3 | 1 | | 0.5 | | 0.3 | | 0.1 | 0.5 |
| Parfum | | | | | | | | | | | |
| PCL-Liquid 100 | | | | 5 | | | | | 2 | | |
| Cetearyl Ethylhexanoate | | | | | | | | | | | |
| PCL-Solid | | | | | | | | 3 | | | |
| Stearyl Heptanoate, Stearyl Caprylate | | | | | | | | | | | |
| Pemulen TR-2 Polymeric Emusifier | 0.3 | | | | | | | | | | |
| Acrylates/C10-30 Alkyl Acrylate Crosspolymer | | | | | | | | | | | |
| Plantacare ® 2000 UP | | | 15 | | | | | | | | |
| Decyl Glucoside | | | | | | | | | | | |
| Potassium Sorbate | | | | 0.2 | | | | | | | |
| Potassium sorbate | | | | | | | | | | | |
| Propylene Glycol | | 2 | | | | | | | | 5 | |
| Propylene Glycol | | | | | | | | | | | |
| Retinopeptide 189 | 1 | | | | | | | | | | |
| Glycerin, Pentylene Glycol, Aqua, Myristoyl Nonapeptide-3 | | | | | | | | | | | |
| Salicylic Acid | 0.3 | | | | | 0.1 | 0.3 | 0.2 | | | |
| Salicylic Acid | | | | | | | | | | | |
| Shea Butter (Organic) | | | | | | | | 20 | | | |
| Butyrospermum Parkii (Shea) Butter | | | | | | | | | | | |
| Sodium Benzoate | | | | 0.2 | | | | | | | |
| Sodium Benzoate | | | | | | | | | | | |
| Sodium Chloride | | | | 6 | | | | | | | |
| Sodium Chloride | | | | | | | | | | | |
| Sodium Hydroxide 10% solution | 2.43 | | | | 2 | 0.58 | 0.46 | | | | |
| Sodium Hydroxide, water | | | | | | | | | | | |
| Softigen ® 767 | | | 3 | | | | | | | | |
| PEG-6, Caprylic/Capric Glycerides | | | | | | | | | | | |
| Solubilizer | | | | | | | 1.2 | | | 2 | |
| PEG-40 Hydrogenated Castor Oil, Trideceth-9, Propylene | | | | | | | | | | | |
| Glycol, Water (Aqua) | | | | | | | | | | | |
| SymCalmin ® | | | | | | | | | | 0.5 | |
| Butylene Glycol, Pentylene Glycol, Hydroxyphenyl | | | | | | | | | | | |
| Propamidobenzoic Acid | | | | | | | | | | | |
| SymClariol ® | | 0.3 | 0.5 | | | | | 0.3 | | | |
| Decylene Glycol | | | | | | | | | | | |
| SymDecanox HA | | | | | | | | 2 | | | 0.5 |
| Caprylic/Capric Triglyceride, Hydroxymethoxyphenyl Decanone | | | | | | | | | | | |
| Symdiol ® 68 | | 0.5 | 0.8 | | | | 0.5 | | 0.5 | 0.5 | |
| 1,2-Hexanediol, Caprylyl Glycol | | | | | | | | | | | |
| SymHair ® Restore | | | | | | | | | | 0.5 | 1 |
| Glycerin, Triticum Vulgare Protein, Aqua | | | | | | | | | | | |
| SymHair ® Shield | | | | | | | | | | | 0.5 |
| Pentylene Glycol, Aqua, Glycerin, Triticum Vulgare Bran Extract, | | | | | | | | | | | |
| 1,2-Hexanediol, Caprylyl Glycol | | | | | | | | | | | |
| SymMatrix | | | | | | | 0.3 | | | | |
| Maltodextrin, Rubus Fruticosus (Blackberry) Leaf Extract | | | | | | | | | | | |
| SymMollient ® S | | | | | | | | | 2 | | 2 |
| Cetearyl Nonanoate | | | | | | | | | | | |
| SymMollient ® W/S | | 2 | 1.5 | | | 2 | | | | 3 | |
| Trideceth-9, PEG-5 Isononanoate, Water (Aqua) | | | | | | | | | | | |
| SymOcide ® PS | | | | | | 1 | | | | | 0.8 |
| Phenoxyethanol, Decylene Glycol, 1,2-Hexanediol | | | | | | | | | | | |
| SymRelief ® S | | | | | | | | 0.1 | | | |
| Bisabolol, Hydroxymethoxyphenyl Decanone | | | | | | | | | | | |
| SymSave ® H | 0.5 | 0.5 | | | 0.5 | | | | 0.5 | | |
| Hydoxyacetophenone | | | | | | | | | | | |
| SymSitive ® 1609 | | | | | 1 | | | | | | |
| Pentylene Glycol, 4-t-Butylcyclohexanol | | | | | | | | | | | |
| SymSol ® PF-3 | | 1.5 | | | 3 | 1.2 | | | | | |
| Aqua, Pentylene Glycol, Sodium Lauryl Sulfoacetate, Sodium | | | | | | | | | | | |
| Oleoyl Sarcosinate, Sodium Chloride, Sodium Oleate | | | | | | | | | | | |
| SymVital ® AgeRepair 3040 | | | | | 0.2 | | | | | | |
| Zingiber Officinale (Ginger) Root Extract | | | | | | | | | | | |
| White Pigment | | | | | | | | 7 | | | |
| CI77891, Ricinus (Castor) Seed oil | | | | | | | | | | | |
| Witch Hazel-Distillate | | | | | | | | | | 1 | |
| Hamamelis Virginiana (Witch Hazel) Water, Water (Aqua), Alcohol | | | | | | | | | | | |
| Xiameter ® PMX-200 Silicone Fluid 100 cs | 1 | | | | | | | | 0.5 | | |
| Dimethicone | | | | | | | | | | | |
| Xiameter ® XM OFX-0193 Fluid | | | | | | 1 | 1 | | | | |
| PEG-12 Dimethicone | | | | | | | | | | | |
| Yellow Pigment | | | | | | | | 0.15 | | | |
| CI77492, Triethoxycaprylylsilane | | | | | | | | | | | |

| | |
|---|---|
| Water | Ad 100 |
| Aqua | |

12 = Pore Refining Fluid
13 = Make-Up Remover Wipes Solution for impure skin
14 = Anti-acne Cleansing Mousse
15 = 3-Phases Clear Make-up Remover Lotion for oily skin
16 = Eau micellaire
17 = Purifying/Anti-Imperfections Cocktail
18 = Tightening Serum for young skin
19 = Concealer Stick
20 = Hair Mask
21 = Aqueous-based Hair & Scalp Serum
22 = Hair Conditioner

1. A Tetraselmis suecica extract comprising the following based on the extract dry weight:
a) total minerals ≥10 wt. % of the total composition,
b) mannitol ≥5 wt. % of the total composition,
c) total galactose, which is the sum of free and bound galactose, ≥3 wt. % of the total composition,
d) total glucose, which is the sum of free and bound glucose ≥4 wt. % of the total composition,
e) total amino acids ≥3 wt. % of the total composition,
f) total nitrogen ≥2 wt. % of the total composition.

2. A Tetraselmis suecica extract according to claim 1 comprising the following based on the extract dry weight:
a) total minerals 11 to 25 wt. % of the total composition,
b) mannitol 6 to 15 wt. % of the total composition,
c) total galactose, which is the sum of free and bound galactose, 4 to 15 wt. % of the total composition,
d) total glucose, which is the sum of free and bound glucose, 4 to 10 wt. % of the total composition,
e) total amino acids 4 to 10 wt. % of the total composition,
f) total nitrogen 3 to 5 wt. % of the total composition.

3. Method of obtaining a Tetraselmis extract comprising the step of extracting a cell material comprising viable, freeze-dried or dried cells of Tetraselmis, with a liquid extractant selected from the group consisting of 2-propanone, ethanol, water, methanol, isopropanol and mixtures of two or more of these extractants, and wherein the extraction comprises:
a) exposition of the cell material to the extractant for up to 8 h at a temperature higher than 60° C., and b) removal of the cell material to obtain the extract.

4. A method according to claim 3, wherein the Tetraselmis extract comprises the following based on the extract dry weight:
a) total minerals ≥10 wt. % of the total composition,
b) mannitol ≥5 wt. % of the total composition,
c) total galactose, which is the sum of free and bound galactose, ≥3 wt. % of the total composition,
d) total glucose, which is the sum of free and bound glucose ≥4 wt. % of the total composition,
e) total amino acids ≥3 wt. % of the total composition,
f) total nitrogen ≥2 wt. % of the total composition.

5. Method according to claim 3, wherein the Tetraselmis classification is Tetraselmis sp.

6. A combination composition comprising Tetraselmis extract according to claim 1 further comprising niacinamide.

7. Combination composition according to claim 6, wherein the weight ratio range of Tetraselmis extract to niacinamide is 1:10000 to 1:1, wherein all weights are calculated based on dry weights.

8. A Tetraselmis extract concentrate comprising:
a) 0.5 to 80 wt. % Tetraselmis extract according to claim 1, calculated based on dry weights,
b) 0.5 to 90 wt. % water,
c) 0.5 to 90 wt. % liquid carrier,
d) optionally 0.1 to 5 wt. % of one or more preservative or preservative system.

9. A liquid Tetraselmis extract concentrate comprising:
a) 0.5 to 10 wt. % Tetraselmis extract according to claim 1, calculated based on dry weights,
b) 30 to 70 wt. % water,
c) 20 to 50 wt. % glycerin,
d) 5 to 20 wt. % 1,2-pentanediol,
e) optionally 0.1 to 5 wt. % of one or more preservative or preservative system.

10. Solid Tetraselmis extract concentrate comprising:
a) 0.5 to 10 wt. % Tetraselmis extract according to claim 1, calculated based on dry weights,
b) 0.5 to 8 wt. % water,
c) 50 to 98 wt. % solid carrier.

11. A method of treating or preventing dysfunctions of human hair and/or skin, seborrhoeic dermatitis, acne vulgaris, wound healing, tissue regeneration, postinflammatory hyperpigmentation, inflammatory related diseases, dandruff or Pityriasis versicolor comprising administering to a subject in need thereof a Tetraselmis extract according to claim 1.

12. A method of treating a skin disease comprising administering a dermatological or therapeutic product comprising a Tetraselmis extract according to claim 1 and optionally auxiliary substances.

13. Non-therapeutic or cosmetic product comprising a Tetraselmis extract according to claim 1 and optionally auxiliary substances and/or perfumes, wherein the cosmetic product is a skin and/or hair care product.

14. Cosmetic product according to claim 13, wherein the amount of Tetraselmis extract or Tetraselmis extract concentrate calculated based on dry weights, in the product is 0.0001 to 10 wt. %.

15. A method of using a Tetraselmis extract according to claim 1 for application on, caring, cleansing, sun-protecting or protecting the skin or for reduction of sebum.

16. A method of using a Tetraselmis extract according to claim 1 for;
a) stimulation of cutaneous junctions,
b) stimulation of cutaneous antimicrobial peptides,
c) reduction of COX-2 gene expression and prostaglandin mediated effects,
d) reduction of post-inflammatory hyperpigmentation,
e) stimulation of filaggrin.

17. A method of using a Tetraselmis extract according to claim 1:
a) for improvement of epidermal integrity of the skin,
b) for prevention of external stimuli such as air pollution or particulate matter induced effects,
c) for prevention of skin barrier dysfunction.

18. Cosmetic product according to claim 13 further comprising one or more of the following:
other sebum reducing agents,
anti-acne agents,
anti-dandruff agents,
other anti-inflammatory agents,
TRPV1 antagonists,
anti-itch agents,
anti-microbial agents, especially anti-Propionibacterium acnes agents,
anti-Malassezia agents.

19. (canceled)

20. A method of using the cosmetic product according to claim 18 for:
a) application on, caring, cleansing, sun-protecting or protecting the skin or for reduction of sebum,
b) stimulation of cutaneous junctions,
c) stimulation of cutaneous antimicrobial peptides,
d) reduction of COX-2 gene expression and prostaglandin mediated effects,
e) reduction of post-inflammatory hyperpigmentation,
f) stimulation of filaggrin,
g) improvement of epidermal integrity of the skin,
h) prevention of external stimuli such as air pollution or particulate matter induced effects; or
i) prevention of skin barrier dysfunction.
